(19) United States
(12) Patent Application Publication
(10) Pub. No.: US 2026/0028574 A1
(43) Pub. Date: Jan. 29, 2026

(54) INCUBATOR AND METHOD

(71) Applicant: EPPENDORF SE, Hamburg (DE)

(72) Inventors: Wolf Lukas Hellweg, Hamburg (DE); Philipp Kayser, Hamburg (DE); David Bondesson, Hamburg (DE); Vjaceslavs Hrupins, Hamburg (DE); Sebastian Maurer, Hamburg (DE)

(21) Appl. No.: 19/100,038

(22) PCT Filed: Aug. 1, 2023
(86) PCT No.: PCT/EP2023/071277

§ 371 Date: Jan. 30, 2025

(30) Foreign Application Priority Data
Aug. 2, 2022 (EP) 22188395.2

(51) Int. Cl. C12M 1/34; C12M 1/00; C12M 3/00

(52) U.S. Cl. CPC C12M 41/36; C12M 23/48; C12M 41/14

(57) ABSTRACT

The invention relates to an incubator for living cell cultures, a method of working with an incubator and a system with an incubator. An image capture system is used to determine an occupancy of the incubator using a mathematical comparison operation.

The invention relates to a system with an incubator for living cell cultures and a method for working with the system.

Such incubators are used in biological and medical laboratories to keep cells in cell culture under controlled environmental conditions, thus enabling the growth of living cells in vitro. For this purpose, the temperature and the gas composition or humidity of the atmosphere inside an incubator chamber isolated from the environment are kept at the desired values by the incubator's equipment. Eukaryotic cells require CO₂ incubators. The atmosphere is formed by air with a certain CO₂ and O₂ content and a certain humidity; a suitable temperature is often 37° C.

The growth of the cells depends critically on the consistency of the atmospheric conditions in the incubator. Disturbances in the incubator atmosphere may comprise a negative effect on cell growth. In an “ideally” equipped laboratory in this respect, each individual user would be provided with a separately accessible incubation chamber for each sample to be incubated. However, this is not realistic for reasons of cost efficiency. In practice, it is common for laboratories to comprise a single incubator (or a few incubators) with a single incubation chamber and one or more storage areas on one or more bearing plates in the incubation chamber for use by multiple users.

The frequency with which the chamber door of the incubator is opened and thus the interference with the controlled atmosphere of the incubator scales with the number of users and also with the number of samples incubated there. The intensity of the disturbance also depends on the duration of the door opening. The more time a user needs to access the interior of the incubator chamber, the longer the door remains open.

There are various usage scenarios for the incubator that may require increased access time due to complications:

### Scenario A) Placing New Objects in the Incubator

If a user places one or more objects, in particular cell culture containers, in the incubator, they need a free storage place in a storage area. If no free storage place is accessible due to disorganized storage, the user needs time to create this storage place; the more carefully he moves or rearranges objects already present in the incubator (stock objects), possibly even documenting this in writing, the more time-consuming the process becomes. If it turns out that there is no longer sufficient storage place available in the incubator chamber, the user repeats the procedure in another compartment of the incubator chamber or in any replacement incubator in the laboratory. This extends the period of time in which the incubator door is open, i.e. the duration of exposure of the chamber interior to the environment (exposure time).

### Scenario B) Testing Cell Cultures

If a user checks the cell cultures he has previously placed in the incubator, e.g. to assess the quality of the cell medium or the state of growth, the user will first search for the relevant cell culture container in the incubator. This extends the exposure time. The more carefully the user moves or rearranges stock items, the more time-consuming the search process becomes.

Scenario C) Removing the Objects from the Incubator

In this case too, the user must first search for the relevant object. The time-delaying factors mentioned in B) apply.

In addition, the more frequently an incubator is opened, the higher the risk of contamination of the interior. There are also cases, for example in forensics or reproductive medicine, in which the value of a single sample, especially cell(s) in a cell culture vessel, is much higher than, for example, the value of the entire incubator, so that contamination-related loss of the sample must be avoided at all costs. In any case, the frequency of contamination increases the risk of downtime, increases costs and requires additional maintenance. After contamination of an incubator, the chamber must be cleaned and sterilized before the incubator can be used again. During this time, if no replacement incubator is available, work with cell cultures is interrupted.

In laboratories, there is therefore a fundamental need to keep the period during which the incubator door is open as short as possible and also to minimize the frequency with which the incubator chamber is opened. For this purpose, an incubator with an image capture system for detecting the storage areas in an incubator and for outputting information on the occupancy status of the incubator was described in the subsequently published European patent application with the application Ser. No. 21/153,810. This functionality makes it possible to limit user access to what is absolutely necessary and to minimize the duration of exposure, i.e. the period of time during which an incubator door is open, and thus the duration of exposure of the interior of the incubator chamber to the environment of the incubator.

The present invention is a further development of this approach.

The task underlying the present invention is to provide a solution for making an incubator efficiently usable, in particular for minimizing the risk of contamination in an incubator.

The invention solves this problem by the system according to claim 1, and the method according to claim 15. Preferred embodiments are in particular objects of the subclaims.
The system according to the invention for monitoring an occupancy of storage place in at least one incubator for incubating living cell cultures, comprises:
at least one incubator for the incubation of living cell cultures, comprising
an incubator chamber for receiving objects, in particular cell culture containers, which comprises opposite inner walls and a chamber opening for the supply and removal of the objects by a user, and which comprises at least one storage area for storing the objects, which extends between the opposite inner walls,
an incubator door to close the chamber opening,
an image capture system comprising
   a lighting device,
   at least one camera device and
   a data processing device, in particular with a data storage device,
   wherein the image capture system is configured to, in particular with the incubator door closed or open.
   to illuminate by means of the lighting device the at least one storage area extending between the inner walls,
   to capture by means of the camera device at least one image of the at least one storage area extending between the inner walls in the form of image data, and
   preferably to store the at least one image by means of the data processing device in the form of the image data in the data storage device;
wherein the system comprises a data storage apparatus and a programmable data processing apparatus programmed thereto:
   to determine at least one occupancy value from the image data, which characterizes the occupancy of the at least one storage area;
   to perform at least one mathematical comparison operation that compares the at least one occupancy value with at least one occupancy reference value;
   to record the result of the at least one mathematical comparison operation in at least one occupancy evaluation parameter; and
   to store the at least one occupancy evaluation parameter in the data storage apparatus.

The image capture system makes it possible to take images in the incubator chamber under controlled conditions and in a reproducible manner, which can provide information about the occupancy of the storage area that can be used in a variety of ways. Since the incubator according to the invention provides information about the occupancy of the storage area in the form of image data, users are able to retrieve information about the occupancy status of the incubator before opening the incubator. By providing this information, unnecessary opening of the incubator is reduced and the use of the incubator becomes more efficient.

The processing of information about the occupancy of the storage area can consist of providing the user with an image of the storage area, for example by displaying it on a display of the incubator. In this way, the user can immediately get a “picture” of whether the occupancy of the storage area allows the storage of further objects, and whether the corresponding storage area contains an object that has been stored by the user and is now to be inspected or removed-insofar as this is identifiable for the user- and where this could be found. The information can also be further processed by using automated image analysis to obtain data on free storage place in this storage area and communicating this to the user. Recognition of object classes and individual object features is also made possible by the availability of the image data.

The incubator can be used even more efficiently by providing occupancy evaluation data that can be carried out automatically.

An occupancy value is, for example, the area size of a sub-area of at least one storage area, wherein this sub-area may comprise a predefined shape, preferably rectangular.

Such a sub-area can be determined from the image data using image processing algorithms, in particular segmentation methods. The sub-area can correspond to a storage location.

An occupancy reference value is a predetermined area value stored in the data storage device that can be used for the mathematical comparison operation. The choice of occupancy reference value depends on the context of the application.

A mathematical comparison operation is a computer algorithm that outputs the result of a mathematical comparison “less than”, “equal to” or “greater than”. This result is stored as an occupancy evaluation parameter. The value is preferably a Boolean, i.e. the Boolean data type used in programming to represent the logical truth values true and false. However, the result of the comparison can also comprise more than two possible values, in particular a classification of a size value carried out by means of a multi-level comparison. This can be used to determine the area size class. This can be typical for certain laboratory sample containers or can be standardized. A sub-area whose area is smaller than that of a first size class can simultaneously fall into a second size class with a smaller area (than the area of the first size class). Such a sub-area may be identified as suitable to serve as a storage location of a laboratory sample container falling within the second size class.

Such an occupancy evaluation can in particular be related to the observation of one, in particular exactly one, storage area in the incubator, and can in particular be related to the evaluation of the free storage place, also referred to as storage location, for a single laboratory container requiring a footprint. In particular, the occupancy evaluation can reduce the risk that a user, who in particular individually evaluates the image display of a storage area on an incubator monitor with regard to occupancy, opens the incubator door due to an individual misjudgment, to deposit a laboratory container in a supposedly free storage place, but this free storage place turns out to be too small for the laboratory container, so that the door opening turns out to be unnecessary, which leads to an additional exposure time for the samples already incubated in the incubator, which increases the risk of contamination for the incubator interior.

Such an occupancy evaluation can in particular be related to the observation of one or more than one storage area, in particular several or all storage areas of an incubator, and can in particular be related to the evaluation of the free storage place for more than one laboratory container requiring a footprint. The occupancy evaluation can be used to evaluate the occupancy or free capacity of at least one storage area. The occupancy evaluation can be used to determine the number of free storage locations and/or the number of occupied storage locations.

In order to assess whether one or more storage places are free (and/or occupied) in the (entire) free storage area of a storage area, it can be determined in particular whether areas of one or more area components of the free storage places are larger than a comparison area, namely in particular the footprint desired for one or more storage locations, which can include at least one predetermined size and at least one predetermined shape.

Images of the storage area can be used to determine the free/occupied storage place. Images of the storage area taken from a bird's eye view are particularly easy to evaluate. Free or occupied storage place can be easily determined using computer-aided image processing, in particular using algorithms for image segmentation. In this way, the utilization of the incubators in particular can be tracked. Based on the detection of free/occupied individual storage locations, capacity statistics can be determined. On this basis, laboratory managers can optimize resource planning and utilization, in particular plan the processing of a large number of laboratory samples using more than one incubator or even more than one laboratory, each with one or more incubators or systems according to the invention. A system according to the invention is hereinafter sometimes also referred to as a “system” for short.

The detection of a free/occupied storage location can be accompanied by the data processing apparatus being programmed to assign a storage location ID to the detected storage location in the form of storage location ID data. The storage location ID data can contain information about the position (storage location position data) of the individual storage location in a storage area of an incubator, wherein this storage area can also be identified by a storage area ID, and wherein the incubator can be identified by an incubator ID. The information about the storage area ID and/or the incubator ID can also be part(s) of the storage location ID data. Storage location ID data can be used to make the use of one or more incubators in the system according to the invention even more efficient. In this way, the user not only receives information about the presence of a free storage place in the incubator and/or the number of certain free/occupied storage locations, but also information about whether an individualized storage location is free or occupied. Storage location ID data can be a component of occupancy status data, which is described below.

The storage locations of an incubator differ from each other in certain characteristics. For example, laboratory containers placed close to the incubator door are easier to access, but may be moved and shifted more frequently by other users than laboratory containers located at the back of the chamber. Since a particular goal in laboratories is the reproducibility of experiments and work processes, the use of individualized storage locations helps to record and store precise information about the individual storage location of the biological sample in a laboratory container and thus make it available for further use, in particular for logging or statistical analysis. Advantageous planning can then in particular provide for designating individualized storage locations for users who have certain class characteristic(s), e.g. storage locations near the back wall, which are better protected from frequent access by other users than the storage locations near the door. Samples that need to be inspected more frequently are preferably positioned closer to the door than to the rear wall of the chamber. Using a positioning guidance system for the laboratory containers, the user can then be efficiently guided to position a laboratory container in a previously recognized and predetermined free storage location. A positioning guidance system that works in particular by means of lighting is described below.

Individual storage place occupancy detection can be identified as follows: A storage location is uniquely identified by storage location ID data. A free storage place is defined as an area of a free storage location that fulfills at least one predetermined criterion: in a storage area, a contiguous area with a predetermined shape, e.g. rectangle, square, circle, and a size that exceeds a predetermined minimum area size SPmin and optionally does not exceed a predetermined maximum area size SPmax: this means in particular that a storage location has a predetermined size, but does not necessarily have to have a predetermined position in the storage area—although this is also possible and preferred. Classes of storage locations may be defined, which may in particular correspond to sizes of commercially available cell culture vessels and their classes. An individual storage location can be uniquely identified by storage location ID data. Storage location ID data does not necessarily have to enable localization in the storage area. It can also simply be a link to the incubator (incubator ID) and/or the storage area (storage area ID) of the incubator. The prerequisite in each case is preferably the determination of at least one free storage location, which can be determined by comparison with a reference value in accordance with the occupancy evaluation.

The preferred embodiments and further developments of the system according to the invention and its occupancy evaluation described here are preferably implemented by programming the data processing apparatus to execute the corresponding functions. This circumstance of the realization of a function is explained once by way of example: to implement individual storage place occupancy detection, the data processing apparatus is preferably programmed to assign storage location ID data to a storage location previously detected by means of image evaluation, which uniquely identifies this storage location.

Image processing segmentation techniques, which can be used to implement computer-aided image analysis to detect a free or occupied storage place/storage location, are known from many image processing applications. Segmentation is based on image processing or image evaluation of individual image data or video image data. Such segmentation methods can be implemented using relatively simple means such as suitable cameras and image processing algorithms. The theoretical principles and their use for the practical realization of segmentation are well known (e.g: “Clustering Techniques for Image Segmentation”, Siddiqui, Yahya, Springer International Publishing, 2021). Ready-to-use image processing algorithms for segmentation are also freely available (OpenCV.org) and well documented. OpenCV (short for Open Computer Vision) is a free program library (BSD license) with algorithms for image processing and computer vision. The OpenCV program library also contains functions, libraries and interfaces for the use of function libraries for segmentation, in particular OpenCV, cv2, matplotlib, numpy, scikit-image.

An occupancy evaluation can in particular be related to the observation of one or more than one storage area, in particular several or all storage areas of one or more incubators of a system according to the invention, and can in particular be related to the evaluation of the free storage place for more than one laboratory container requiring a footprint.

In particular, an occupancy evaluation can relate to the observation of one or more than one storage area, in particular several or all storage areas of one or more incubators of a system according to the invention. The occupancy evaluation may compare a footprint corresponding to more than one storage location for a laboratory container with a predetermined occupancy reference value for this situation. The occupancy evaluation can compare a (total) free storage place of one or more storage areas of one or more incubators of a system according to the invention with a predetermined occupancy reference value for this situation. In particular, the occupancy evaluation then does not determine the number of available or occupied individual storage locations, even if this is possible in principle. Preferably instead, information is obtained and provided as to whether free storage place falls below/exceeds a predefined minimum/maximum value (“/” here means “and/or”). For example, if the (total) free storage place is greater than a predefined maximum value, information can be obtained about reaching/falling below a minimum capacity utilization. If the free storage place is less than a specified minimum value, for example, information can be obtained about reaching/exceeding the minimum utilization of an incubator. If more than one incubator is included in the consideration of the total free storage place of a system, information can be obtained about reaching/falling below/exceeding utilization limit values in an incubator network. Such a network can be formed by the incubators in one or more laboratory rooms or in a laboratory. In this way, a laboratory manager can gain valuable information on resource planning/utilization in one or more laboratories.

In a preferred application, the occupancy assessment can be used to detect when a minimum utilization is reached or not reached in order to schedule the cleaning of one or more incubators. Since the incubator must be empty during cleaning, the occupancy evaluation then helps to identify the best time to clean an incubator-because the fewer remaining samples need to be removed and moved, the less effort is required. In the case of a network of incubators, the occupancy evaluation helps to select the most favorable incubator for cleaning—the (total) free storage places of the individual incubators are compared with each other and the incubator with the largest free storage place is determined.

The system can be set up to generate incubation report data for an individual sample contained in an individual laboratory sample container (cell culture container). For this purpose, the sample/laboratory sample container is preferably assigned a sample ID or sample ID data by means of programming and computer-assisted operations, with which the sample/container can be uniquely identified. Since the laboratory sample container is arranged in a storage location of the incubator, the sample ID can be assigned a storage location ID (storage location ID data), which is then stored together in the incubation report (in the incubation report data). If the laboratory sample container is tracked using an object tracking system, movement history data can also be assigned to the sample ID data, which can include time-dependent information about the movement history in particular.

Assuming that a laboratory sample container essentially always remains in its predetermined storage location, object tracking is not absolutely necessary to obtain movement data of an individual laboratory sample container. Thus, by monitoring the storage location and time-dependent observation of image changes in this storage location, a measure of the (unwanted) movement of the laboratory sample container can be obtained, which can be stored in the form of movement data. This data can also be assigned to the sample ID and stored in the incubation report data. In particular, time-dependent sensor data obtained from sensors of the incubator relating to a gas concentration inside the chamber (CO₂, O₂, N₂, H₂O) and/or the internal chamber temperature and/or data on the number and duration of door openings can also be assigned to the sample ID data in order to obtain incubation report data. This incubation report data can be used to record the sample treatment in detail and create the basis for a high reproducibility of experiments and sample processing.

The incubator may comprise a camera device, in particular the camera device of the image capture system, by means of which at least one image of at least one storage location of at least one storage area can be captured. In particular, image documentation to be created for an individual storage location identified by means of storage location ID data can be captured and stored as a function of the storage location ID data and/or sample ID data. The image documentation can be video documentation. The activity of the camera device and the image capture can be controlled as a function of a door sensor in order to only record when movement of the laboratory sample container is at least possible. The image documentation can be stored as part of the incubation report data.

The system according to the invention may comprise a positioning guidance system. The positioning guidance system preferably has an illumination device by means of which an area or spot in the storage area, in particular a storage location, can be specifically illuminated. The lighting device can also be formed by the lighting device that is part of the image capture system.

The lighting device is preferably arranged within the incubator chamber in such a way that several areas, in particular all, in particular essentially all, areas of the at least one storage area can be illuminated. Preferably, light is emitted onto the storage area and reflected and/or scattered from there in the direction of the user, i.e. in the direction of the door opening (incubator door).

A data processing device of the incubator, or the data processing apparatus, is preferably programmed to control the activity, i.e. the switching on/off, and/or the illumination color, and/or the illumination intensity, and/or the timing of a pulsed activity of the illumination apparatus.

A data processing device of the incubator, or the data processing apparatus, is preferably programmed to set the target of the illumination as a function of sample ID data and or storage location ID data and to apply directional illumination to that target or storage location. The lighting device is preferably set up to set the target of the lighting and to apply directional lighting to this target or this storage location. For this purpose, the lighting device may comprise an electrically controllable movement device which carries at least one light source, in particular an electrically operated light source (e.g. LED) or at least one light-emitting optical fiber, and which is set up for the targeted illumination of different storage locations.

The lighting device can be arranged on or at a storage area, in particular at/on a storage location, so that the emitted light falls from there into the eye of the observer.

The system according to the invention may comprise a user guidance system. The user guidance system may comprise a positioning guidance system. The user guidance system is set up to support the positioning of laboratory sample containers on and/or between storage locations of the incubator by illuminating at least one storage location and/or a laboratory sample container placed on a storage location in accordance with a predetermined sequence plan (sequence plan data).

Preferably, a data processing device of the incubator, or the data processing apparatus, is programmed for this purpose,
to allow the user to select or enter a sample ID by means of a user interface device,
and/or assign storage location ID data, also referred to as reserved storage location

ID data, to this sample ID according to the predetermined schedule, which in particular determines a duration of incubation of the sample,
and/or to control the lighting of the storage location reserved by predetermination according to a predetermined schedule by means of the lighting device, depending on this sample ID and the reserved storage location ID data.

In particular, storage location position data can also be used, which, similar to object position data, defines the position of a (free or occupied) footprintin a storage area. The storage location data is preferably also part of the storage location ID data.

A footprint preferably has a predetermined size A2 (e.g. measured in square centimeters) and/or a predetermined dimension M, which in particular also contains information about the shape, wherein the rectangular shape can be assumed as the default shape. For example, microtiter plates and cell culture bottles have an essentially rectangular footprint, while Petri dishes and other bottle shapes may comprise a circular footprint. Preferably, different sizes A2 and dimensions are provided which correspond to certain commercially available objects and which can be stored in the data storage apparatus for comparison or automated image evaluation as comparison values or occupancy reference values. Since some free space is also required for practical handling, e.g. an edge of a width d (e.g. 2 cm), this is added in order to be suitable for a footprint that is used for a specific cell culture container. An SBS standard microtiter plate has the dimensions length×width (“outside dimension of the base footprint”) according to the specifications in the ANSI/SBS 1-2004 standard: {127.76 mm±0.5 mm}×{85.48 mm±0.5 mm}. Including a margin d, this would result in approximately M=170×126} and A2=110.08 cm² (without margin) or A2=214 cm² (with margin).

Example of pairings A2, M are commercial products (without border):
Roth Selection cell culture bottle with 175 cm² growth surface: M={20.5 cm×12.0 cm}, A2=246 cm²
Roth Selection cell culture bottle with 75 cm² growth surface: M={15.0 cm×8.5 cm}, A2=127.5 cm²
Roth Selection cell culture bottle with 25 cm² growth surface: M={9.0 cm×5.0 cm}, A2=45 cm²
Tissue Culture Tray, Mitchell Plastics™ M={40 cm×19.0 cm}, A2=760 cm²

The data storage apparatus and/or the programmable data processing apparatus of the system according to the invention can be components of the incubator, i.e. in particular installed within the incubator housing, in which the incubator chamber is also arranged, but they can also be components of an external data processing device, in particular a computer, server or other laboratory device, which is connected to the incubator for the purpose of exchanging data.

The incubator is a laboratory device or laboratory incubator. An incubator refers in particular to a laboratory device with an incubator chamber whose atmosphere can be regulated or is regulated by the incubator to a predetermined target temperature. In particular, it is a laboratory device, with which controlled climatic conditions can be created and maintained for various biological development and growth processes. The incubator can be or contain a shaking incubator (shaker), i.e. an incubator with a movement device for moving objects arranged in the incubator chamber, and can be a microbial incubator (also without CO₂). In particular, the incubator can be designed as a cell cultivation device. The incubator is used in particular to create and maintain a microclimate with controlled gas and/or humidity and/or temperature conditions in the incubator chamber, wherein this treatment can be time-dependent. The laboratory incubator, in particular a treatment device of the laboratory incubator, can in particular comprise a timer, in particular a timer, a heating/cooling device and preferably a setting for the regulation of an exchange gas supplied to the incubator chamber, an adjusting device for the composition of the gas in the incubator chamber of the incubator, in particular for adjusting the CO₂ and/or the O₂ and/or the N₂ content of the gas and/or an adjusting device for adjusting the humidity in the incubator chamber of the incubator. The incubator, in particular a treatment device of the incubator, has in particular the incubator chamber, furthermore preferably a control device with at least one control circuit to which the at least one heating/cooling device is assigned as an actuator and at least one temperature measuring device is assigned as a measuring element. The temperature in the incubator can be controlled by means of the control device. Depending on the embodiment, the humidity can also be controlled. A tray filled with water in the incubator chamber can be heated or cooled in order to adjust the humidity via evaporation. Alternatively and/or additionally, a water evaporator can be provided as part of the incubator, by means of which the humidity in the atmosphere of the incubator chamber is adjusted. CO₂ incubators are used in particular for cultivating animal or human cells. Incubators may comprise turning devices for turning the at least one cell culture container and/or a shaking device for shaking or moving the at least one cell culture container. In particular, the incubator according to the invention is not a bioreactor or fermentor.

The incubator may comprise a sensor device. In particular, a sensor device comprises at least one temperature sensor, preferably a plurality of temperature sensors. A temperature sensor can be a Pt 100 or Pt 1000 temperature sensor, for example. A sensor device preferably has a sensor for determining a relative gas concentration, in particular for determining the content of CO₂ and/or O₂ and/or N₂. A sensor device preferably has a sensor for determining the relative humidity.

An incubator preferably comprises one or a single incubator chamber. This can be divided into compartments. Compartments can be separated by—in particular perforated-bearing plates, wherein in particular a gas exchange between the compartments is made possible.

A bearing plate, in particular its lower side, can be designed to hold the camera device and can in particular comprise a holder for the camera device. A bearing plate, in particular its lower side, can be set up to hold the lighting device and can in particular comprise a holder for the lighting device. However, the lighting device or its holder can also be arranged or mounted elsewhere in the incubator chamber, e.g. on an inner side wall of the incubator chamber, or on the floor or ceiling wall. A holder for the lighting device may comprise a rail system, a robotic arm controlled by the control device and/or (a) magnet(s).

The incubator chamber has chamber walls or chamber inner walls and exactly one or at least one chamber opening, via which the objects or cell culture containers can be placed inside the incubator chamber and removed. This chamber opening can be closed by a closure element movably connected to the incubator chamber, in particular an incubator door movably mounted on the incubator chamber by means of a hinge, in particular one or more chamber doors. An incubator may comprise one or more inner doors, which may in particular be transparent, and may comprise an outer door, in particular a non-transparent outer door, which in particular thermally insulates the incubator chamber and possibly at least one inner incubator door, which closes or opens the chamber opening, from the environment. Preferably, the image capture system captures images when the incubator door or outer door is closed so that ambient light does not influence the illumination of the storage area, which is preferably provided exclusively by the lighting device. This leads to particularly reproducible, easily comparable image recordings that are simple to evaluate using image processing algorithms. Nevertheless, it is also possible for the images to be taken with the incubator door open.

In the closed position of the chamber opening, the interior of the incubator chamber is preferably insulated from the environment in such a way that a desired temperature or atmosphere controlled by the incubator can be set, in particular regulated, in the interior.

In the open position of the chamber opening, gas exchange between the environment of the incubator and the interior of the incubator chamber is possible via this opening. The chamber opening is typically located in a front wall surrounding the chamber opening.

The incubator chamber preferably comprises several walls or inner wall surfaces, which can in particular be connected to each other in one piece and in particular without edges. The walls or inner wall surfaces are preferably essentially planar in shape, but may also comprise a curved shape in whole or in part. The incubator chamber is preferably rectangular in shape, but can also be shaped differently, e.g. spherical, ellipsoidal, polyhedron-shaped. The walls or inner wall surfaces are preferably made of a low-corrosion material, in particular stainless steel, copper, brass, or a plastic, in particular a composite plastic. This makes it easier to clean/disinfect the inside of the chamber. Irrespective of the chamber opening, which is used to load/remove objects or cell culture containers, the incubator chamber may comprise at least one port for passing an appropriately dimensioned device or a cable connection from the inside of the incubator chamber to the outside of the incubator or into the environment of the incubator.

Preferably, the surfaces of the incubator inner walls are non-glossy or non-reflective, in particular by using a matt surface. The surface of the incubator inner wall can be matted by a surface treatment. The surface treatment can in particular be grinding with an abrasive, which can in particular comprise a specific grain size. The surface treatment can in particular be blasting with a blasting agent, in particular sand or glass beads, in particular using compressed air, which can in particular comprise a specific grain size or a characteristic particle diameter. This can prevent or reduce interfering reflections in an image recording.

A typical size of the interior of an incubator chamber is between 50 and 400 liters.

The incubator may comprise exactly one incubator chamber, but can also comprise several incubator chambers whose atmosphere (temperature, relative gas concentration, humidity) can be adjusted individually or collectively. An incubator may comprise several incubator chambers, each of which may comprise its own chamber opening and its own chamber door for closing the chamber opening.

The incubator may comprise a housing that partially or completely surrounds the incubator chamber. The housing can be essentially cuboid in shape and can in particular be designed in such a way that the incubator can be stacked.

A storage area of the incubator is realized in particular by a bearing plate, in particular a shelf plate insert and/or a moving platform, which may in particular be made of stainless steel or copper or the like or comprise this material. A bearing plate serves as a base plate, in particular as an intermediate base plate. The bearing plate can be removable from the incubator chamber (“bearing plate insert”) or can be permanently attached to it. The incubator chamber may comprise holding sections or a holding frame for holding one or more bearing plate inserts or insertable instruments. The underside of a bearing plate can be designed to hold a camera, in particular a holder for this camera. Alternatively or additionally, at least one of the inner walls of the incubator chamber can be designed to hold one or more bearing plate inserts or insertable instruments, in particular the at least one camera. For this purpose, a holding structure integrated into the wall can be provided, in particular one or more protrusions, grooves or bars. A bearing plate increases the available storage space in the incubator chamber.

Preferably, essentially all surfaces or at least one surface of the at least one bearing plate are non-glossy or non-reflective, in particular by using a matt surface. The surface of the inner wall of the incubator can be matted by a surface treatment. The surface treatment can in particular be grinding with an abrasive, which can in particular comprise a specific grain size. The surface treatment can in particular be blasting with a blasting agent, in particular sand or glass beads, in particular using compressed air, which can in particular comprise a specific grain size or a characteristic particle diameter. This can prevent or reduce interfering reflections in an image recording.

A holding frame for the at least one bearing plate is also preferably made of a non-corrosive material, preferably stainless steel. The holding frame is preferably designed as a standing object in that it comprises at least one base section which rests on the floor wall of the incubator chamber. However, it can also be supported on the side walls of the incubator chamber and/or suspended from the ceiling wall of the incubator chamber.

A bearing plate preferably—and in particular essentially completely-extends over a horizontal cross-section of the incubator chamber.

Preferably, an incubator comprises at least two bearing plates that are arranged one above the other. The volume area between two bearing plates or between a bottom wall of the incubator chamber and a lowest bearing plate or between a ceiling wall of the incubator chamber and an uppermost bearing plate can be referred to as a storage compartment. A storage compartment can be understood as a storage area as a whole. The surface of a bearing plate suitable for storage can be regarded as a storage area. The height of a storage compartment is preferably dimensioned so that an object of a certain maximum height (measured perpendicular to the planar surface of a bearing plate) or a stack of objects of a certain maximum height of the stack can be placed on the bearing plate. In particular, the maximum height can essentially correspond to the distance between two bearing plates.

The distance between two bearing plates or the maximum height is in particular between 5 cm and 70 cm, preferably between 5 cm and 65 cm, preferably between 5 cm and 60 cm, preferably between 5 cm and 50 cm, preferably between 10 cm and 30 cm, preferably between 10 cm and 20 cm, preferably between 12 cm and 18 cm. In particular, the maximum height can be up to 150 cm. The distance between two bearing plates is preferably selectable by the user by means of a variable holding device for bearing plates.

An instrument that can be inserted into the interior of the incubator chamber, in particular a camera, can be designed as a module and enables automated observations to be made inside, preferably even when the incubator door is closed.

The camera device, or at least one camera thereof, is preferably arranged on a bearing plate, preferably arranged or arrangeable below a bearing plate and in particular attached or attachable thereto. Preferably, at least one camera is mounted or mountable on the underside of a bearing plate, in particular in a geometric center of the underside, in particular at the intersection of the diagonals of a rectangular underside.

Preferably, one camera- or several cameras—is mounted or can be mounted on the underside of a shelf insert in the incubator chamber, or on an underside of the upper inner wall (ceiling wall) of the incubator chamber, preferably in each case vertically above the geometric center of the storage area that the camera monitors. However, one or more cameras can also be arranged or mounted or can be arranged/mounted on the inner side walls of the incubator chamber or on a holding frame.

Preferably, the at least one camera is set up and arranged such that it comprises an image angle, generally measured in the image diagonal or alternatively also in the image vertical or image horizontal, which is between 90° and 210°, preferably between 120° and 180°, and preferably between 160° and 180°.

A camera of the image capture system may comprise a wide-angle lens, in particular a fisheye lens, whose image angle in the image diagonal can be between 120° and 230°.

Preferably, exactly one camera is provided on the underside of a bearing plate, which comprises in particular one of the above-mentioned angles of view.

The “field of view” or field of view (FOV) of a camera can be defined in particular as comprising a certain image angle, e.g. an image angle according to one of the ranges defined above, which leads to the display of an image content dependent on this angle of view, or can be defined by an image angle measured in the image vertical and an image angle measured in the image horizontal. The aspect ratio of the image can in particular be one of the following formats: 4:3, 3:2, 16:9, 1:1.

Preferably, at least one camera-preferably exactly one camera—is arranged on the underside of a bearing plate, the field of view of which preferably covers more than X % of the storage surface of a bearing plate located in the field of view of the at least one camera. X is preferably 20, 30, 40, 50, 60, 70, 80, 90, 100 in each case. In other words, the image captured by this-precisely one or at least one-camera preferably shows more than X % of the storage surface of a bearing plate located in the field of view of the at least one camera. For example, several cameras can be provided which together capture the entire storage area, i.e. 100% of the storage surface, or the X proportion. Preferably, exactly one camera is provided, which captures the entire storage area or part X. The larger or more complete the field of view, the more reliably or efficiently the storage area can be mapped and the image evaluated.

Preferably, at least one camera-preferably exactly one camera—is arranged on the underside of a bearing plate, the viewing area of which is located in a compartment of the incubator and which, preferably in addition to the storage surface (a part or the entire storage surface) on the bearing plate, preferably captures more than Y % of the wall surface of a compartment wall bounding the compartment, which is formed by an inner wall section of the inner wall of the incubator. Y is preferably 20, 30, 40, 50, 60, 70, 80, 90, 100, respectively. In other words, the image captured by this-precisely one or at least one-camera preferably shows more than Y % of the wall area of one (or all) of the compartment bounding compartment walls. For example, several cameras can be provided which together capture the entire surface of all compartment walls, i.e. 100% of the inner wall surface of a compartment, or the proportion Y. Preferably, exactly one camera is provided that captures the entire surface of all compartment walls, or the Y portion. The correspondingly large field of view means that objects or stacks of objects positioned at one edge of the planar storage area of the bearing plate can also be detected.

The data processing device is preferably programmed to automatically crop the image captured by the camera so that an effective image angle is obtained which is smaller than the image angle specifying the camera, or so that an effective field of view is obtained which is smaller than the field of view specifying the camera.

The camera of the incubator is particularly suitable to work reliably in the respective incubator atmosphere over a period of several months or years, or to work reliably during the service life measured under standard conditions (room temperature). Not every camera is suitable to work in an incubator atmosphere. A possible commercially available camera is the 5MP Wide Angle Camera for Raspberry Pi, www.joy—it.net, available from Conrad Electronic SE, Germany, and/or another camera in combination with a wide angle lens, e.g. commercially available the “Industrial lens HAL 250 2.3”, Entaniya Co., Ltd, Japan. Alternatively, an enveloping device can be provided for the at least one camera in order to shield or isolate it from the incubator atmosphere, wherein this enveloping device comprises in particular transparent areas or a transparent window or is transparent in order to enable the image to be taken through the transparent area.

Preferably, the camera device has at least one optical filter with which the light incident on the camera is filtered. This allows the quality of the image capture to be optimized, in particular with regard to downstream digital image processing and evaluation. Preferably, the camera device has at least one polarizing filter with which the light incident on the camera is filtered. This can reduce reflections in the image recording that can arise from reflections of the light of the lighting device on objects in the storage area, elements of the storage area or the incubator chamber and/or inner walls of the incubator chamber, in particular with regard to downstream digital image processing and evaluation. The polarizing filter is preferably a circular polarizing filter, but can also be linear. Unwanted reflections from smooth, non-metallic surfaces (e.g. the plastic surface of cell culture containers) can be suppressed using a polarizing filter. On non-metallic surfaces, light with vertical polarization is noticeably more strongly reflected, especially if the exit angle to the surface is around 30° to 40°, i.e. close to the Brewster angle. If the polarization filter is suitably aligned, the reflected light waves are suppressed so that the unpolarized background is not outshone by the reflections. Preferably, when polarization is used, the objects to be recorded, in particular cell culture containers, are arranged—in particular in a direct line-between the lighting device and the at least one camera.

Preferably, the camera device has a first polarizing filter and the lighting device has a second polarizing filter, wherein in particular the first and second polarizing filters are used rotated in relation to each other. In this way, some of the light from the lighting device is initially blocked out by the polarizing filter in front of it. The polarizing filter in front of the camera is adjusted or rotated in relation to the polarizing filter of the lighting device so that it now also blocks out the other part of the light emitted by the lighting device. Ideally, only diffuse light remains. As a result, reflections, including those from metallic surfaces, are reduced or completely eliminated. This is particularly advantageous with regard to downstream digital image processing and evaluation, especially with regard to outline detection of objects by means of image processing.

The lighting device preferably has at least one light source, in particular an LED. Preferably, the lighting device has at least two light sources or more, each with a different emission spectrum, i.e. different colors, e.g. red, green, blue. In this way, the image quality can be optimized, in particular with regard to downstream digital image processing and evaluation, in particular with regard to outline detection of objects by means of image processing.

The lighting device may comprise at least one light source whose emitted light comprises or consists of wavelengths greater than that of visible light, in particular whose emission spectrum is in the infrared range with in particular a wavelength between 780 nm and 1 mm, in particular in the near infrared (780 nm to 3000 nm) or medium infrared (3000 nm to 50000 nm) or contains such an infrared range. In this case, the camera device has at least one camera or camera sensor that is suitable for detecting corresponding light, in particular infrared light. The lighting device may comprise at least one light source whose emitted light comprises or consists of wavelengths smaller than that of visible light. In this case, the camera device has at least one camera or camera sensor that is suitable for detecting corresponding light.

Preferably, at least two light sources are arranged at a distance from each other. As a result, the field of view of the camera(s) can be illuminated more homogeneously and the intensity of individual areas of reflection dependent on the direction of light can be reduced.

This is particularly advantageous with regard to downstream digital image processing and evaluation, especially with regard to outline detection of objects by means of image processing.

Preferably, the lighting device has at least one light source that is arranged or fastened or can be arranged/fastened to an underside of a bearing plate. Preferably, the lighting device comprises at least two or more light sources, which are arranged or fastened or can be arranged/fastened at different positions along an underside of a bearing plate.

Preferably, the lighting device has at least one optical filter through which the light emitted by the lighting device is partially or completely filtered. The optical filter can be a polarizing filter, which in particular can be matched to a polarizing filter of the camera device in order to achieve the optimum desired filter effect.

Preferably, the lighting device has at least one light diffuser, wherein the lighting device emits a diffuse light. A light diffuser can be, for example, a milky Plexiglas plate or comprise one. The light diffuser can reduce or prevent hard shadows and reflections, which is particularly advantageous with regard to downstream digital image processing and evaluation.

Preferably, the image capture device, in particular the camera device and/or the lighting device, has at least one diaphragm, preferably an aperture diaphragm, which in particular has a variable diameter, e.g. an iris diaphragm, in order to control the light flow of the lighting or the light flow entering the camera.

Preferably, the image capture device, in particular the camera device and/or the lighting device, has at least one or more optical lenses.

Preferably, the image capture device, in particular the camera device and/or the lighting device, has at least one lens, preferably a wide-angle lens, preferably a wide-angle fisheye lens.

Preferably, the image acquisition device has a timer. Preferably, the data processing device is programmed to activate at least one or more light sources of the lighting device in a predetermined time sequence and in particular to deactivate them again after a predetermined activity time in each case and/or to activate all or more light sources simultaneously, and preferably programmed so that the camera device captures several images of the storage area, which are captured one after the other and in particular synchronously with the activity times of the lighting.

Preferably, the image capture system is set up to record and store the time at which an object enters the incubator chamber and/or the time at which an object is removed from the incubator chamber.

Preferably, the image capture system is configured to:
to illuminate at least one or two objects arranged on this storage area by means of the lighting device,
to capture an image of the at least one or two objects on this storage area by means of the camera device, and
to store the image of the at least one or two objects in the data storage device in the form of image data by means of the data processing device. In this way, various possible uses of the image data are made possible, in particular: distinguishing objects in the storage area, in particular: assigning different identification data to the first and second object; counting the objects; recognizing the object class; analyzing, storing and recognizing individual features; tracking objects during movement; recognizing and storing the time of entry or removal of the object.

Preferably, the data processing device is programmed for this purpose,
to distinguish the first object and the second object shown in the image by means of evaluation of the image data, in particular: to assign different identification data to the first and second object; to count the objects; to recognize the object class; to analyze, store, recognize individual features; to track objects in motion, in particular to detect the outlines of the first and second object in the image by means of image processing algorithms, and
in particular to store information about the first and second objects, in particular the bounding boxes and/or outlines of the first and second objects, in the form of object data in the data storage device.

Preferably, in a preferred embodiment, the lighting device is arranged, and in particular the data processing device is programmed, to operate the lighting device in at least two different illumination modes, and the image capture system is preferably arranged, and in particular the data processing device is programmed for this purpose,
the storage area of the incubator chamber by means of the lighting device to illuminate
   i) initially in a first illumination mode and
   ii) then in a different second illumination mode,
to capture at least one image of the storage area during illumination by means of both the first and second illumination modes by means of the camera device, and
to provide the at least one image in the form of image data containing combined image information captured during both the first and second illumination modes, wherein the data processing device is programmed to execute an image evaluation program that extracts the combined image information from the image data.

In particular, this embodiment can improve or optimize the quality of the image capture of the storage area, which is particularly advantageous with regard to downstream image processing, in particular image evaluation, in particular for capturing the bounding box(es) and/or the outline(s) of one or more objects or cell culture containers.

A first illumination mode and a second illumination mode can differ in particular in that different light sources are used, and/or light sources arranged at different positions, and/or different exposure times of the light sources, and/or different emission spectra or light colors, and/or different light intensities. The different illumination modes can in particular improve or optimize the quality of the image capture of the storage area, which is particularly advantageous with regard to downstream image processing, in particular image evaluation, especially for capturing the outline(s) of one or more objects or cell culture containers.

Preferably, the at least one image of the storage area includes at least a first image of the storage area and a second image of the storage area different therefrom, wherein the first image is captured in the first illumination mode and the second image is captured in the second illumination mode, and the first image is provided in the form of first image data and the second image is provided in the form of second image data, wherein in particular the data processing device and/or the image evaluation program are programmed for this purpose
the first image data and the second image data are combined to obtain combined image data, which is obtained in particular by an addition and/or averaging of first and second image data, and
the combined information is obtained from the combined image data.

In particular, this embodiment can improve or optimize the quality of the image capture of the storage area, which is particularly advantageous with regard to downstream image processing, in particular image evaluation, in particular for detecting the positions of one or more objects or cell culture containers in the image of the storage area by means of bounding box algorithms.

A typical program code for image-processing object tracking, which is preferably used, is based on the evaluation of the temporal sequence of images. A typical program code for object tracking uses a “bounding box” as an output format to identify an object in an image, to define its collision boundaries and, in particular, to localize it. In digital image processing, the “bounding box” refers to the coordinates of the rectangular frame that largely or completely encloses an object shown in the digital image. The use of bounding boxes in object tracking makes it more efficient, as image evaluation using such a numerical aid requires fewer computing steps and therefore less computing power, particularly in comparison with algorithms for outline detection of objects. In addition, the corresponding algorithms can be executed efficiently and cost-effectively using specialized graphics processing units (GPUs). Suitable programming interfaces (APIs) for object tracking using bounding boxes are available in the OpenCV program library under the names BOOSTING, CSRT, GOTURN, KCF, MEDIANFLOW, MOSSE, MIL, TLD. Accordingly, OpenCV provides program libraries (“MultiTracker”) for the simultaneous tracking of multiple objects (“multiple object tracking”). Deep learning algorithms for multi-object tracking (MOT) based on the “tracking-by-detection” principle are known as an alternative.

However, it is also possible and preferred that the contour of the object to be tracked in the image is determined for object tracking, and in particular the separation of object (fore-ground) and background by background subtraction.

Preferably, a plurality (N>=10) of illumination modes are used to either capture an image or capture multiple images, which then provide a combined image with combined image information in the form of combined image data. Preferably 2<=N<=300, preferably 10<=N<=300, preferably 100<=N<=300, where preferably N<=500 or N<=1000.

Preferably, the at least one image of the storage area contains a multi-exposed image of the storage area, the image capture system being set up in particular for this purpose,
to expose and capture the image of the storage area during illumination by means of both the first and second illumination modes by means of the camera device, and
to provide the multi-exposed image in the form of image data.

Preferably, the at least one image contains information about objects arranged in the storage area, in particular information, optionally,
via the positions of the objects in the storage area
via the outer contours of the objects,
via the area of the objects, measured in a plane parallel to a planar surface of the storage area,
the area of the storage area not occupied by the objects, measured in a plane parallel to a planar surface of the storage area.

Preferably, the lighting device comprises at least a first and a second light source which are operated differently in the first and second illumination mode, wherein in particular the first and a second light source are arranged at a distance above a storage space of the storage area, wherein in particular the first and second light source are arranged offset in a plane which is parallel to a planar storage space of the storage area, wherein in particular the storage area comprises a planar storage space, wherein the first light source is arranged vertically above a first half of the storage space and the second light source is arranged vertically above a second half of the storage space, wherein in particular the lighting device comprises an LED light strip with several LED light sources, which is arranged in a plane which lies parallel to a planar storage space of the storage area, in particular in a meandering course, a spiral-like course, in particular in an at least partially linear course, wherein in particular the image capture system comprises an electronic control device, in particular a programmable electronic control device, which is set up or programmed in such a way that the LED light strip is arranged in a plane which lies parallel to a planar storage space of the storage area, in particular in a meandering course, a spiral-like course, in particular in an at least partially linear course, wherein in particular the image capture system comprises an electronic control device, in particular a programmable electronic control device, which is set up or programmed in such a way that
during an illumination phase of the first illumination mode, the first light source is operated differently than during an illumination phase of the second illumination mode, and/or
during an illumination phase of the first illumination mode, the second light source is operated differently than during an illumination phase of the second illumination mode, in particular that
during an illumination phase of the first illumination mode, the first light source is active and during an illumination phase of the second illumination mode, it is less active (i.e. emits at a lower intensity) or inactive, and/or
the second light source is less active or inactive during an illumination phase of the first illumination mode and is active during an illumination phase of the second illumination mode, in particular that
during an illumination phase of the first illumination mode, the first light source is operated with a different emission spectrum than during an illumination phase of the second illumination mode, and/or
during an illumination phase of the first illumination mode, the second light source is operated with a different emission spectrum than during an illumination phase of the second illumination mode.

Preferably, in particular, the at least one camera is arranged at a distance vertically above a storage space of the storage area, wherein preferably the at least one camera comprises a wide-angle lens, in particular a wide-angle or fisheye lens, wherein preferably exactly one camera is provided, which is arranged at a distance vertically above a center of the storage space of the storage area.

Preferably, the image capture system is a modular component of the incubator, namely a component which can be optionally used by the user, wherein in particular the incubator comprises a control device and a temperature control device for controlling the temperature in the interior of the incubator chamber, wherein the image capture system comprises another control device which is set up to control the image capture system, in particular in that this other control device includes the data processing device of the image capture system. Such a modular embodiment of incubator with image capture system preferably further comprises a data interface to the incubator so that, for example, image data can be displayed on the incubator display.

Preferably, in particular, the incubator has a control device and a temperature control device for controlling the temperature in the interior of the incubator chamber, in particular this control device being set up to control the image capture system, in particular in that this control device includes the data processing device of the image capture system. This is the integral embodiment of the incubator with image capture system.

Preferably, the incubator has a display and is preferably set up or programmed to show on the display
preferably the image, and/or
preferably image information extracted from the at least one image, and/or preferably display an image of the storage area containing the combined image information.

The image capture system is preferable:
an object detection system in which the data processing device is programmed to detect the at least one object located in the storage area during image capture of the at least one image by means of the image evaluation program,
in particular an object recognition system for recognizing the object on the basis of individual properties and/or for recognizing an object class on the basis of object-specific class properties,
in particular an object tracking system for tracking changes in position of the at least one object in the storage area from a start position to detect its end position.

“Downwards” denotes the direction of gravity, “upwards” the opposite direction. “Vertical” means “along the vector of gravity”, “horizontal” means perpendicular to the vertical or in a planar plane perpendicular to the vertical. When used as intended, incubators are arranged so that the tops of the planar bearing plates are horizontal.

Preferably, the incubator comprises a treatment device for treating the at least one object, in particular a cell culture container. The term “treatment” means in particular that an object, in particular a cell culture or a cell culture container, is moved and/or transported and/or examined and/or modified, in particular physically, chemically, biochemically or otherwise modified.

A treatment device can be a movement device by means of which the cell medium is kept in motion in at least one cell culture container, preferably via a movement program that is controlled by the control program. A movement device can be a shake or swivel device. A movement device preferably has a carrier device, in particular a plate, on which one or more cell culture containers are placed and/or fixed. A movement device preferably has a drive device, in particular in the case of a shaking device, for example an oscillator drive, in particular in combination with an eccentric, by means of which the desired movement program is implemented. A treatment device can be a swivel device by means of which at least one cell culture container is swiveled. The components of the swivel device can correspond to those of the shaking device, but are set up for a swivel movement.

A treatment device can also be a transport device by means of which at least one cell culture container can be transported in the incubator chamber.

The transport device can be a lift device with a carrier device on which at least one object, in particular a cell culture container, camera or light source, can be placed. The transport device or lift device preferably comprises a movement mechanism and/or an electrically controllable drive mechanism for driving the movement mechanism. The transport device can also be a movable and electrically controllable gripping arm for gripping and holding at least one cell culture container. The transport device may comprise a conveyor belt or a rail system for moving the at least one object placed on it. The transport can be used to move the at least one object in the incubator chamber, in particular to a processing position or pick-up position, e.g. in a processing station, in the incubator chamber, and away from this processing position or pick-up position. The control device can be set up to control the transport device as a function of information from previously recorded image data.

A treatment device can also be a transport device by means of which at least one camera of the camera device and/or at least one light source can be transported in the incubator chamber. In particular, the transport device can be arranged below or directly below a bearing plate and/or below or directly below a ceiling wall of the incubator chamber. Different illumination modes can be realized using a moving or movable light source. In particular, the illumination mode can be adapted to an occupancy state, e.g. in order to variably realize a suitable lighting direction when a storage area is very densely occupied with objects. Several cameras, as well as a moving or movable camera, can be used to create different images or image sections of the storage area, which can be combined to create an overall image of the storage area, particularly by means of digital image processing. In the case of a movable camera, it is also possible to adapt the camera position to an occupancy condition, e.g. to variably realize a suitable lighting direction when a storage area is very densely occupied with objects.

In particular, the data processing direction can be programmed to transport the at least one camera of the camera device and/or at least one light source in the incubator chamber by means of the transport device in a predetermined or dynamically adapted manner. For example, the data processing direction can be programmed to transport the at least one camera of the camera device and/or at least one light source in the incubator chamber by means of the transport device to different recording positions, in particular to evaluate the image created there in each case by means of an image processing algorithm and in particular to check whether a desired image information, e.g. an individual feature of a cell culture container, in particular a barcode, has been recorded in sufficient quality, e.g. in order to read the barcode unambiguously. The data processing direction can be programmed to move the camera and/or the light source to other imaging positions until the desired image information has been captured.

The camera and/or lighting device can also be attached to a transport device. The camera and/or lighting device can be or can be attached to a positioning mechanism, by means of which the camera and/or lighting device can be moved and positioned in the incubator chamber. The positioning mechanism can include a movable robot arm and is preferably electrically controllable, in particular by a control program of the control device. In this way, different recording situations can be recorded one after the other with one or a few camera and/or lighting devices. The positioning mechanism can be designed as a component that can be inserted into the incubator chamber. This component can be supplied with power via a cable connection to the incubator, preferably via a cable connection through a wall opening, e.g. a port, or via such a cable connection to an external power source. The control device can be set up to control the positioning mechanism as a function of cell monitoring data.

The treatment device can also be understood as the temperature control device of the incubator chamber, with which the atmosphere inside the incubator chamber is controlled to the desired value, in particular 37° C. The term temperature control refers to raising and lowering the atmospheric temperature by heating and cooling. Preferably, the temperature inside is adjusted by changing the temperature of the walls of the incubator. Temperature sensors of the corresponding temperature control device are distributed at at least one position inside and/or outside the incubator chamber, in particular on a wall of the incubator chamber.

The incubator preferably comprises a user interface device via which the user can input data to the data processing device or the control device and/or via which information can be output to the user. Preferably, the incubator or this user interface device is set up so that the user can enter at least one operating parameter for operating the incubator or the image capture system at this user interface device or can receive information from it. In this way, a single user interface device can be used by the user to influence or control, or receive information from, the incubator and also the at least one image capture system. In particular, the image capture system can be set up to display position data or free storage place to the user in response to a query made by the user via the user interface device of the incubator, or to display information derived from position data (e.g. identity of the user who caused the position change), in particular also statistical information, such as, for example, frequency and time of the position change of an object (a sample) and/or—in particular as a percentage-available free storage place, and/or at least one optical image of the at least one object, in particular with or without the storage area. This is advantageous for the user, as this information provides him with essential information which, on the one hand, allows him to plan experiments more precisely-before he carries out an experiment, he knows that footprint is available; on the other hand, the change in position of samples, particularly in the first few hours after seeding of adherent cells, has a negative effect on their adhesion; no uniform cell lawn is then formed. The provision of information on position changes and their frequency in accordance with the invention allows the user to determine the causes of uneven cell growth and thus to take these into account in future experiments.

A device-controlled treatment of the incubator is preferably a program-controlled treatment, i.e. a treatment controlled by a program. A program-controlled treatment of an object is understood to mean that the treatment process is essentially carried out by processing a plurality or number of program steps. Preferably, the program-controlled treatment is carried out using at least one program parameter, in particular at least one program parameter selected by the user. A parameter selected by a user is also referred to as a user parameter. The program-controlled treatment is preferably carried out by means of the digital data processing device, which is in particular a component of the control device. The data processing device or the data processing apparatus may comprise at least one processor, i.e. a CPU, and/or may comprise at least one microprocessor. The program-controlled treatment is preferably controlled and/or carried out in accordance with the specifications of a program, in particular a control program. In particular, in the case of program-controlled treatment, essentially no user activity is required, at least after the program parameters required by the user have been recorded. Device-controlled treatment of the incubator can be carried out in particular as a function of previously acquired image data. Image capture by the image capture system is in particular a program-controlled treatment, namely imaging of the storage area or object.

The data storage apparatus or the data storage device preferably comprises at least one data memory, which may in particular be a volatile or a non-volatile data memory. The data captured or received by the incubator can be stored on this at least one data memory, in particular in at least one database, which can be stored in at least one data memory. This data includes, in particular, at least one or all of the following types of data: image data, still image data, video image data, object data, combined image data, first and second image data, identification data, ID location data, user identification data, user-related ID location data, object identification data, movement history data, class-related ID location data, individual-related ID location data, occupancy status data, in particular storage location ID data. The data storage device/data storage apparatus is preferably part of the incubator, i.e. in particular arranged in a housing of the incubator. However, it can also be part of an external data processing device with which the incubator or its data processing device communicates. The data storage apparatus is a component of the system according to the invention.

A program parameter is a variable that can be set in a predetermined way within a program or subroutine, valid for at least one execution (call) of the program or subroutine. The program parameter is defined, e.g. by the user, and controls the program or subroutine and causes a data output depending on this program parameter. In particular, the program parameter and/or the data output by the program influences and/or controls the control of the device, in particular the control of the treatment by means of the at least one treatment device.

A program or program code or computer program code is understood to mean, in particular, an executable computer program. This is stored in a data memory or on a data storage medium. A program is a sequence of instructions, in particular consisting of declarations and instructions, in order to be able to process and/or solve a specific functionality, task or problem on a digital data processing device. A program is usually available as software that is used with a data processing device. In particular, the program may be present as firmware, in the case of the present invention in particular as firmware of the control device of the incubator or the system. The program is usually present on a data carrier as an executable program file, often in so-called machine code, which is loaded into the working memory of the computer of the data processing device for execution. The program is processed as a sequence of machine, i.e. processor, instructions by the computer's processor(s) and thus executed. The term “computer program” also refers in particular to the source code of the program, from which the executable code can be generated in the course of controlling the laboratory device.

A user interface device can be a component of an incubator or a module. A user interface device preferably has in each case: a control device for the user interface device; a communication device for establishing a data connection with a laboratory device, in particular an incubator, via an interface device of the same; an input device for recording user input from a user; an output device, in particular a display and/or a display, for outputting information to the user, in particular a touch-sensitive display. The control device of the user interface device is preferably set up to exchange data with the control device of the incubator via the data connection.

An object is in particular a cell culture container. A cell culture container is particularly transparent. In particular, it is made of plastic, especially PE or PS, and in particular comprises a planar base plate which forms the growth surface for the cells. This may comprise a surface treatment to promote the adherence of cells. The cell culture container can be closed or provided with a PE cap or gas exchange cap, in particular a lid with an optional filter. The cell culture container is particularly stackable. An Eppendorf cell culture bottle is particularly suitable. The object can be a stack of cell culture containers, in particular a stack of Petri dishes or cell culture bottles.

Preferably, the data processing device is programmed to detect (time-dependent) changes in the appearance (or the appearance) of the objects from one or more images, in particular between longer time intervals of minutes, hours or days. In this way, color changes of the cell culture medium or colors in a cell culture container or structures, e.g. droplets, on a cell culture container wall can be determined. Such colors, color changes or structures can indicate problems in the respective cell culture, e.g. a nutrient deficiency, pH value changes, or mold, or other contaminations. Preferably, the data processing device is programmed to output information to the user or operating personnel via a user interface depending on the detection of the appearance of a cell culture container or these changes in the appearance of the cell culture container and/or to store the data about this detection (in particular: what was detected and when) in a data memory and to make it available for retrieval.

Image-processing object tracking techniques are well known, for example when used in drones or driver assistance systems for tracking vehicles or people. Object tracking is based on image processing or image analysis of video image data. Such object tracking methods can be implemented using relatively simple means such as suitable cameras and image processing algorithms. The theoretical principles and their use for the practical realization of object tracking techniques are well known (e.g: “Fundamentals of Object Tracking”, S. Challa et al., Cambridge University Press, 2011). Immediately usable image processing algorithms for object tracking are also freely available (OpenCV.org) and well documented. OpenCV (short for Open Computer Vision) is a free program library (BSD license) with algorithms for image processing and computer vision. The OpenCV program library also includes functions for tracking multiple objects in real time. The use of object tracking in incubators has not yet been published and represents an innovation.

A typical mode of operation of image-processing object tracking, which is preferably also used in the object tracking system according to the present invention, is based on the evaluation of the temporal sequence of images. A typical program code for object tracking uses a “bounding box” as an output format to identify an object in an image, to define its collision boundaries and, in particular, to localize it. In digital image processing, the “bounding box” refers to the coordinates of the rectangular frame that largely or completely encloses an object shown in the digital image. The use of bounding boxes in object tracking makes it more efficient, as image evaluation using such a numerical aid requires fewer computing steps and therefore less computing power, particularly in comparison with algorithms for outline detection of objects. In addition, the corresponding algorithms can be executed efficiently and cost-effectively using specialized graphics processing units

(GPUs). Suitable programming interfaces (APIs) for object tracking using bounding boxes are available in the OpenCV program library under the names BOOSTING, CSRT, GOTURN, KCF, MEDIANFLOW, MOSSE, MIL, TLD. Accordingly, OpenCV provides program libraries (“MultiTracker”) for the simultaneous tracking of multiple objects (“multiple object tracking”). Deep learning algorithms for multi-object tracking (MOT) based on the “tracking-by-detection” principle are known as an alternative.

However, it is also possible and preferred that the contour of the object to be tracked in the image is determined for object tracking, and in particular the separation of object (fore-ground) and background by background subtraction.

The performance potential of an object tracking system is based, on the one hand, on reliable automatic identification of an object in the incubator in various typical incubator usage scenarios, which are described below. On the other hand, the approach is efficient because no special adaptations are required on the part of the object. In particular, the object does not have to contain any passive (code, labeling) or active (e.g. a transmitter) identification aids. Instead, the usual objects (cell culture containers, devices, etc.) can be used with the incubator, in particular regardless of manufacturer and external appearance. In particular, the incubator according to the invention is able to distinguish objects with a completely identical appearance by tracking.

Possible scenarios when changing the occupancy in an incubator are:
I. Setting new objects
II. Removing of objects
III. Door is opened and objects are only moved without removing or repositioning one.

### Subconditions:

i) Object(s) are moved
ii) Object(s) are not moved
iii) Several objects are set/removed (sequence).

Assumption: all cell culture containers look the same on the outside. The question underlying the development of the invention was, in particular, which image-based methods could be considered, especially whether still images are sufficient to enable object identification in common usage scenarios of an incubator.

For scenario I. (new object is to be placed in the incubator chamber), it is initially assumed that before the incubator door is opened, there is a current still image of the storage area in the incubator chamber taken by a camera placed in the incubator, which does not yet show the new object.

If the new object is placed in the incubator chamber without moving the existing objects (objects already arranged and localized in the storage area) (case I.ii)), the new object can be identified (without any problems) via the next still image (after closing the incubator door). Object tracking is not necessary for case I.ii). The same applies to Il.ii): if an object is removed, it can be clearly identified by evaluating the still images before and after the door is opened.

If the new object is set and existing objects are moved in the process (case I.i)), the new object cannot be clearly identified via the next still image. Location information about the already registered stock objects is lost. The same applies to the removal of an object and the subsequent moving of the existing objects (case II.i)). The concept of object tracking is used for moving (condition i)).

If several objects (case iii)) are set under condition i), i.e. without moving the existing objects, the new objects can be easily identified using before and after still images, but the information about the setting sequence is lost. If you want to obtain this information, you need object tracking. The same applies if several objects are removed in case i)+iii).

Since moving stock items is the rule rather than the exception when operating an incubator, it is not sufficient in this case to evaluate the before and after images of the storage area.

One particular question when developing an object tracking system in an incubator is: When is an object identified, i.e. at what time or event is identification data assigned to the object? In most application scenarios (apart from a case such as iii)+i), where it may be necessary to record the order in which objects are set), it is sufficient to collect this identification data when the new objects have been set and any existing objects have been moved. This is because the moving of already registered stock objects is carried out under the object tracking measure. In the next freeze frame, i.e. in particular when the incubator door is closed, the new objects can then be registered using the freeze frame. If it is de-sired to record the sequence, the moment an object first enters the camera's field of view, i.e. the first time it appears in an image captured by the camera (a start image, which in this case is a video image), it is registered, i.e. the object is assigned an ID number, and this object is tracked until it reaches its final position; the possible movement of stock objects or their removal is preferably also tracked.

In another practical scenario, the storage area (or several storage areas) is assumed to be occupied by one or more objects (stock objects), which are registered using an initial start screen (in this case, for example, a still image). Only the possible movement of these stock objects needs to be tracked here. The movement of newly set objects does not need to be tracked here during the setting process, as they can be registered again in the next still image. The corresponding presence of these new objects in the video data can there-fore be ignored. In this scenario, the information about the order in which several objects are adjusted during a door opening is lost, but this information is not absolutely necessary.

Accordingly, for a preferred embodiment, the invention proposes to implement object tracking to ensure the correct localization of objects, as needed, in various or all situations.

In particular, the data processing device is programmed to assign identification data to the at least one object introduced into the interior. In particular, this means that a new object is detected in the image data (still images or video data) of the camera. In particular, a new object is detected when it is moved into the camera's field of view from the outside. When the object is detected, it can be assigned identification data on the one hand and position data on the other. The position data, in particular the start and end position of an object, is determined in particular with reference to an internal coordinate system, which is also used to define the position of the at least one storage area and thus also the position of the at least one object in relation to the at least one storage area. This position information is particularly important if the position of the at least one object in the at least one storage area or in the incubator chamber is to be graphically illustrated to the user on a display.

Identification data may be or include an identification number, and/or may include an identification code consisting of any characters or information. This identification data can be predetermined, randomly generated or specified by a user, in particular as long as it is suitable for clearly distinguishing the new object placed in the incubator chamber from the identification data of the other stock objects. The identification data can also be predetermined and merely selected. In the latter case, the term “assign” also includes the creation of new identification data.

In particular, the data processing device is programmed to determine the start position of the at least one object from the start image of the storage area. The start image is prefer-ably a still image that is recorded in a still image mode of the camera. It can also be a single image obtained from video data, in particular a video frame. In particular, the data processing device is programmed to define an enveloping line figure, preferably a rectangle, or an enveloping body, or in particular a bounding box of the object, or an outer contour of the object, in the start image and, in particular, to define the object as the area covered by the enveloping line figure, in particular by the bounding box or an outer contour.

In particular, the data processing device is programmed to determine the changes in position of the at least one object by evaluating the video data. In particular, the data processing device is programmed to track the movement of the object defined in the start image by means of the bounding box. In particular, the data processing device is programmed to detect the movement of the image area containing the object defined in the start image by means of the bounding box by determining the position changes of this image area from frame to frame. In particular, tracking of the bounding box can be used to determine the image area that changes position due to the object movement. In particular, video data contains information that can be used to reconstruct the individual images (“frames”, which are displayed at a certain number per time, i.e. “frame rate”, when the video is displayed) that characterize the video. In the case of uncompressed video data, the latter can also contain the complete sequence of image data, wherein a “set” of image data represents a single image. In the case of compressed image data, temporal changes of pixels of the camera image may also/only be recorded.

In particular, the data processing device is programmed to determine the starting position of the object in the storage area from the starting image. The start position can be deter-mined in particular by the fact that the object, which was previously motionless in a first frame of an image series, shows a change in position of the object in the following frame.

The first frame in which the object comprises a change in position compared to the previous frames can be defined as the start frame. Since the movement of the object begins at a time T1 and ends at a time T2, an image (still image or video image, also an image obtained from superpositioned images) that is captured before the time T1 can be used as the start image, from which the start position of the at least one object is determined.

In particular, the data processing device is programmed to determine the final position of the at least one object in the final frame of the storage area from the position changes. The end position can be determined in particular by the fact that no more position changes of the object are determined from frame to frame. The first frame in which the object comprises no more position changes compared to the previous frames can be defined as the end frame. Since the movement of the object begins at a time T1 and ends at a time T2, an image (still image or video image, also an image obtained from superpositioned images), which is captured from time T2, can be used as the final image, from which the final position of the at least one object is determined. In particular, the end position can be determined by the time at which the door sensor detects the closing of the incubator door. The end position can be determined in particular by the fact that an arm or hand of the user entering the image area is no longer detected. For example, an image can be evaluated to determine whether a section, e.g. strip-shaped, located in the image border area corresponds to a reference state in which a reference section of the incubator chamber or the incubator is completely visible. If this is not the case, it can be concluded that a user is still handling inside the incubator and the object or several objects are still being moved, so that video image capture and evaluation in particular must be continued. The end position of the object can be understood as the position at which the object no longer shows a change in position after previous position changes, and can therefore be determined by the end of the movement of the object. Alternatively or additionally, the end position of the object can be defined in such a way that the position that the object has when the closing of an incubator door is detected by the door sensor is the end position. As a result, the end position will be the same in most cases.

The data processing device is preferably programmed to start capturing the start image and/or video data using the camera when a sensor detects activity occurring at the incubator. The sensor can be a motion sensor that detects movement in a detection area out-side the incubator. The sensor can be a touch sensor that detects a user touching the incubator, e.g. a door handle of the incubator. The sensor can be a door opening sensor that detects in particular the opening of an incubator door, especially an outer door of the incubator. The sensor can be an external camera of the incubator, which uses image analysis to detect movement and/or a person in the camera's field of vision. The sensor can be a proximity sensor that detects the approach of a person to the incubator, e.g. by detecting the change in an electric field.

The data processing device is preferably programmed to start capturing the final image and/or stop capturing video data when a sensor detects an activity taking place at the incubator. The sensor can be a door opening sensor, which in particular detects the closing of an incubator door, especially an outer door of the incubator. The sensor can be an external camera of the incubator, which uses image analysis to detect the end of a movement and/or the disappearance of a person in the camera's field of vision. The sensor can be a proximity sensor that detects the removal of a person from the incubator, e.g. by detecting a change in an electric field.

In particular, the data processing device is programmed to start the acquisition of video data by means of the camera initiated by the opening of an incubator door detected by the door sensor. Alternatively or additionally, an initial event sensor, in particular a motion sensor, or proximity sensor, or optical sensor/receiver (e.g. light barrier), or microphone, or light barrier), or microphone, or acceleration sensor in the incubator door, can be arranged in the incubator, by means of which the approach of an object to the incubator chamber or another initial event can be detected; The trigger for initiating the camera and video data can also be a code entry at a door lock of the incubator, which can be carried out in particular by the data processing device without using measurement results from one of the aforementioned sensors; the data processing device can be programmed to start the acquisition of video data based on the data from such a sensor. The data processing device may be programmed to start searching for a new object in the frames or still image available by means of the video data when the video data and/or a still image is available. Alternatively, the acquisition of video data can start as soon as a user has been identified at the incubator or when another predefined event occurs. Permanent video data acquisition is also possible. In particular, the data processing device is programmed to stop capturing video image data when the final image is captured or when the absence of a hand/arm in the camera field of view is registered, or based on the results of one of the aforementioned sensors (door sensor, motion sensor, etc.).

In particular, the data processing device is programmed to assign identification data to the at least one object in the storage area and to determine the position of the at least one object as ID position data and store it in the data memory. In particular, the data processing device is programmed to store the final position of the at least one object in the storage area as ID position data in the data memory as a function of the identification data of the at least one object. With this step, the incubator “knows” the object and its position. Together with other data, it can now output this data to a user, in particular show it on a display of the incubator. Together with data about the owner (definition owner: the user who has placed the object in the incubator chamber) of the object or a user of the object (e.g. a user who has moved a stock object belonging to another user), the incubator can store and collect these data records depending on the identification data of the object. The identification data on the basis of which the position changes were detected does not have to be identical to the identification data stored as ID position data; what is relevant is that the stored identification data is suitable for clearly distinguishing the at least one object from other objects or inventory objects. The ID code can therefore theoretically change during image processing.

The assignment of an owner to an object can be done in different ways. Preferably, the data processing device is programmed to register or identify the user placing the object in the incubator, to assign a user identification code to this user, and to store user-related ID position data of the object. For a registration, a biometric recognition, in particular face recognition, speech recognition and/or voice recognition, of the user can be carried out, in particular by means of an external camera, a retina scanner or a fingerprint sensor of the incubator. The corresponding registered biometric recognition data, in particular facial recognition data of the user, can be stored in the data storage device of the incubator or in an external data storage device. The user can be identified by comparing the recorded biometric recognition data with previously registered biometric recognition data. As an alternative to biometric recognition, a user can also be enabled to enter user identification data via a user interface device before, during or after performing object registration or after determining the final position of a tracked object. The user interface device can be a keyboard, a touch screen and can be part of the incubator or an external device, or a user name/identifier can be entered by voice input.

One advantage of object tracking is that it can generally be carried out without knowledge of the individual or class characteristics of the object to be tracked. However, it can also be combined with methods for object recognition (and recognition again) and/or object class recognition or class recognition. This is particularly helpful if several objects are tracked in parallel using an object tracking system.

In particular, object recognition can be designed as individual object recognition and/or object class recognition. The theoretical principles and their implementation for practical application in object recognition technologies are well known (e.g: “Deep Learning in Object Detection and Recognition”, X. Jiang et al., Springer Singapore, 2019). Algorithms for object recognition in images are generally known and available, e.g. as part of OpenCV (for example OpenCV 3.3, deep neural network (dnn) module).

Individual object recognition is based on the recognition of individual object characteristics (individual object features), which make it possible to recognize the individual object and distinguish it from other individual objects. For example, a cell culture container, e.g. a disposable product, may comprise subsequently applied individual features, e.g. a bar-code or QR code. However, it can also be identifiable via any features that enable differentiation: e.g. labeling, different contents, a micro-scratch pattern on the container surface, etc.,

Object class recognition is based on the knowledge of object class characteristics, which are compared during the object inspection in order to assign a class to the object. For example, object class recognition can be used to recognize whether an object is a certain type of cell culture bottle, a certain type of Petri dish or a certain type of microtiter plate, possibly taking into account other class characteristics, e.g. manufacturer, year of manufacture, design, etc.,

The incubator preferably comprises an object recognition system. The object tracking system is preferably also set up for object detection.

In the case of object individual recognition, a data processing device of the object recognition system or the object tracking system is preferably programmed a) to recognize individual features of at least one object from a still image, the start image, the video data and/or the end image, and b) to store these individual features of the object in the form of object individual data, in particular as a function of identification data. Preferably, the data processing device is programmed to extract individual object features of the at least one object from the start image, the video data and/or the end image, to compare the individual object features with an individual object database and, if the individual object features in the individual object database are linked to an individual object identifier, to store the individual object features in the form of individual object data, in particular as a function of identification data: identify the object individual identifier of the at least one object; or, if the object individual characteristics in the object individual database are not linked to an object individual identifier: assign an object individual identifier to the at least one object and store it in the object individual database, and/or assign the recognized object individual identifier to the ID position data of the at least one object and store it as individual-related ID position data. An object individual identifier is preferably different from its identification data; however, the object individual identifier can also preferably be the same as its identification data.

In the case of object class recognition, a data processing device of the object recognition system or the object tracking system is preferably programmed a) to recognize class features of the at least one object in a still image, the start image, the video data and/or the end image, and b) to store these class features of the object in the form of object class data, in particular as a function of identification data. Preferably, the data processing device is programmed to recognize object class features of the at least one object in a still image, the start image, the video data and/or the end image, to compare the object class features with an object class database (which in particular contains previously known correlations between the object class and object class features) and to recognize the object class of the at least one object, and in particular to assign the recognized object class to the ID position data of the at least one object as object class data and in particular to store it as class-related ID position data.

Preferably, the incubator has a user identification device by means of which a user using the incubator can be identified in the form of user identification data. Preferably, a data processing device of the incubator is programmed to identify a user using the incubator by means of the user identification device and to assign user identification data to the user and to store identification data and/or ID position data in the data memory as user-related identification data and/or user-related ID position data depending on the user identification data.

Preferably, the user identification device comprises an external camera, and preferably the user identification device is arranged and/or the data processing device is programmed to perform facial recognition by means of the external camera, by means of which the user is identified. Preferably, a user database is provided, which is stored on a data storage device, which may be part of the incubator, the user identification device or the object tracking system, or which may be in a data exchange connection with the user identification device or the data processing device, e.g. via an intranet or the Internet. Algorithms for face recognition in images are generally known and available, e.g. as part of OpenCV (“FaceRecognizer class”).

The user database can contain a correlation of user identification data and user feature data, so that the user or the user identification code (user identification data) can be determined on the basis of the determined or read—in user feature data. The user feature data can contain information about the user's facial features or other biometric data, e.g. finger-print data or voice recognition data. The user database can contain a correlation of user identification data and user identifiers, wherein the user identifier can be a personal identification code of the user, e.g. a multi-digit character string, which a user can identify him-self by entering on a keyboard of the user interface device.

In particular, the external camera can be arranged on, above or next to an incubator door, especially an external door on the incubator, or attached to it. Preferably, the external camera is an integral part of the incubator or the incubator door. However, it can also be connected to the user identification device or the data processing device via a signal connection, in particular via a data exchange connection, which can be wired or wireless. For example, it is possible to connect the external camera to the incubator or its user identification device or the data processing device via a flexible cable, so that the camera can be freely placed on the incubator by the user.

Preferably, the user identification device comprises a user interface device by means of which user identity data can be read in. The user interface device can include a keyboard and/or a touch screen and/or a microphone for voice input or for implementing user identification by means of voice recognition. The user interface device can be set up to ex-change data with an external data-processing device (hereinafter also referred to as “ex-ternal device”). The external device can be a PC, a smartphone, a tablet computer or another portable computer with a user interface.

The external device may comprise means to identify and/or authenticate a user. In particular, currently available smartphones contain various means for user authentication, especially for facial recognition. The external device preferably has software, e.g. an app, which is programmed to identify and/or authenticate a user and, in particular, to transmit the result of this process to the user identification device of the incubator via the user interface device. Since an external device often comprises its own camera, which can be used to implement facial recognition, or a fingerprint sensor, or other hardware for user identification and authentication, the corresponding hardware components can be dispensed with if the incubator is connected to the external device on the incubator.

The user identification device of the incubator can be programmed as a component of a control software of the incubator. The incubator preferably has a control device, which may in particular comprise a data processing device, which may in particular be programmed to include all or some functions of the user identification device, in particular to control the data exchange with the external device.

Preferably, the user identification device comprises a user interface device by means of which user identity data can be selected. In particular, the user identification device may comprise a display or a touchscreen via which a list of possible users can be displayed, e.g. by entering a name or image of the user. Input means can then be provided, e.g. keys, a keyboard, touchpad, the touchscreen, via which the user can make the selection from the list.

The user identification device may be programmed to perform user authentication by pass-word-protecting said reading of user identity data or said selection from the list, so that the user is not considered identified until authentication is successful.

Preferably, the user identification device has a reader for reading a code that identifies the user, wherein the reader is in particular an RFID reader, a barcode reader or a QR code reader.

The user identification device or data processing device can be programmed to unlock and/or lock a locked incubator door depending on the user identification, in particular to unlock a locked incubator door if the user has been successfully identified. In this case, this means that the user is also authorized to access the incubator. However, there may also be an additional access rights list which the incubator uses to decide whether an identified user has access rights or, if applicable, what type of access rights the identified user has. The access right can, for example, be restricted to certain times, in particular weekdays, times or authorization periods. If the incubator comprises several incubator doors, the access right can stipulate that the user only has the access right for a predetermined selection of these incubator doors.

Preferably, the incubator comprises exactly one- or even several-incubator door(s) for closing the chamber opening. When closed, the incubator door forms in particular a part of the incubator housing, which serves as a thermal insulator of the incubator chamber of the incubator. The incubator door may comprise a user interface device on its outside, in particular a display. A data processing device of the incubator or the user interface device may be programmed to display an image of the at least one storage area of the incubator taken by the camera of the incubator.

Preferably, the incubator has a door sensor for detecting the opening or closing of the incubator door. Preferably, the incubator has a motion sensor or a proximity sensor for detecting a person approaching the incubator. Preferably, the data processing device is programmed to start the monitoring of the interior of the incubator, in particular the generation of the video data/still image data, depending on the detection of a door opening of the incubator and/or the approach of a person; Preferably, the data processing device is programmed to start the monitoring of the interior of the incubator, in particular the generation of the video data/still image data, in particular depending on the detection of a door opening by a user who has been identified by means of a user identification device; Preferably, the data processing device is programmed to terminate the monitoring of the interior of the incubator, in particular the generation of the video data, depending on the detection of a door closure of the incubator;

Preferably, the data processing device is programmed to use the information from the user identification device and the object tracking device to determine which user has moved which object in the interior and to store the user identification data of this user together with the object identification data of this object in the data memory.

Preferably, the data processing device is programmed to determine the movement path of the at least one object within the incubator chamber from the start image, the video data and/or the end image and to store it in the data memory in the form of movement history data, in particular to store it in a time-dependent manner. Preferably, the data processing device is programmed to determine a movement history of the at least one object within the incubator chamber from the start image, the video data and/or the end image and to store it in the data memory in the form of movement history data, in particular to store it in a time-dependent manner, preferably with information about the number and/or times of the changes in the status of the door opening (open/closed) of the incubator door deter-mined by means of the door sensor. The movement path preferably contains stored position data of the object, wherein this position data marks the movement path of the object, in particular between a start image and an end image, in particular between a start position of the object—in particular a stationary or even moving position- and an end position—in particular a stationary position. The movement history data preferably contains time-de-pendent stored position data or movement paths, preferably within at least one time period or during the entire stay of this object in the incubator. Movement history data can also include information on the user triggering the position change in the form of user identification data. This is particularly advantageous for objects containing valuable samples.

Preferably, the incubator comprises a display (=a screen). The screen is preferably an integral part of the incubator, in particular the incubator door. However, it can also be arranged remotely from the incubator and can in particular be part of an external device which can be connected to the data processing device of the incubator for data exchange.

Preferably, the data processing device is programmed to display a graphical representation of the interior of the incubator chamber, in particular the at least one storage area, on the screen. The graphical representation may include a photograph of the storage area, on which one or more stock objects of the incubator may be displayed. The storage area may in particular be a bearing plate in the incubator or a predetermined section thereof.

The photo can show an image taken with the camera, which can be post-processed if necessary. Preferably, this post-processing involves straightening a distorted image taken by the camera. Algorithms for such post-processing are generally known and freely available (for example: OpenCV, “Omnidirectional Camera Calibration”). The distortion can be caused by optical factors in particular and can be due to the use of wide-angle or fisheye optics.

In particular, the graphical representation can be an abstracted representation of an image or image section captured by the camera. For example, the graphical representation can be an abstracted storage area shown from a bird's eye view (or another perspective), in particular the graphical representation of a rectangle or the perspective representation of a cuboid. The stock objects can also be represented in an abstracted form, e.g. as rectangular or cuboid graphic image objects. The aim of such a representation is in particular to inform the user about the location of the object(s) in the incubator or storage area. This allows the user to quickly access the desired object(s) and minimizes the time the incubator door is open. If it is necessary to differentiate between individual objects contained in a stack of objects, a graphical representation from a perspective other than a bird's eye view is useful, for example from a lateral perspective, in order to be able to graphically highlight individual objects in a stack.

Preferably, the data processing device is programmed to graphically display where the object identified by the object position data is positioned in the storage area or interior of the incubator chamber, or to graphically display where all objects localized in the interior are located.

Preferably, the data processing device is programmed to graphically highlight one or more objects in the display depending on at least one condition parameter. The condition parameter can designate the user identification data. Highlighting is possible both in an abstracted representation and in a photographic reproduction of an image or image section of the storage area captured by the camera in the incubator display.

Preferably, the data processing device is programmed to graphically highlight an object or several objects on the display which is assigned to the user as the owner, depending on the user identification data of a user (individual user, a user group or several users), for example by the user-related ID position data containing the user identification data of this user. The owner is the person who looks after the object and—in most cases himself or with the help of an assistant—has placed it in the incubator chamber. Preferably, the data processing device is programmed to determine, based on predetermined user identification data, where the objects assigned to this user identification data by means of the user-related object position data are positioned and, in particular, to graphically highlight these objects.

The condition parameter can also contain information about a time period or a point in time, e.g. the length of time an object has already been in the incubator chamber. This allows a user to quickly obtain an overview of how long one or more objects have been stored in the incubator chamber, possibly forgotten by their owner. Alternatively, the incubator can graphically highlight one or more objects that require the attention of the user or laboratory staff depending on an event detected by a sensor in the incubator or depending on a schedule that may be stored in the incubator or an external device.

Or, in the case of the implementation of object class recognition, the condition parameter can contain information about a specific object class. In this way, one or more objects of the same object class (or of different object classes) can be graphically highlighted, for example to emphasize the location of all Petri dishes (and not: cell culture bottles) in the incubator interior.

Or, in the case of the implementation of object individual recognition, the condition parameter can contain information about a specific object individual. In this way, an object search based on individual characteristics can be implemented, for example by the incubator comprising means for entering individual characteristics, e.g. a barcode, QR code, individual labeling or a photo of the individual object. The individual object can thus be graphically highlighted and easily found.

Preferably, the data processing device is programmed to display on the screen a graphical representation of the interior of the incubator chamber, in particular of the at least one storage area, and in particular to graphically display or highlight the free storage place available in the incubator. For example, the storage area can be shown in abstracted form and a free storage position (or several available free storage positions) can be graphically highlighted by displaying the corresponding area, for example, in green or white, or a time-varying (flashing), contrasting color to the background. In this way, the user does not have to spend time searching for a possible free storage location or creating one by moving stock objects.

In addition, similar to the function of a storage attendant, the data processing device can be programmed to plan the occupancy of the interior of the incubator chamber or the at least one storage area and, in particular, to optimize the use of the available storage space in this way. For this purpose, the data processing device may be programmed to take into account predetermined distances between one or more stock objects and a new object to be stored and, in particular, to suggest this to the user by displaying the free storage place as available and/or unavailable. According to these examples, the incubator may comprise a computer/software-implemented planning program for occupying the interior of the incubator, which in particular takes into account the position of at least one object in the interior (inventory object) and/or in particular the freely available storage place, possibly also the times at which the at least one inventory object was newly placed, or times in the future at which further objects are planned to be placed in the incubator. Such points in time can be known in particular if the incubator is connected to a laboratory information system (LIS) or another (laboratory) data exchange network. The incubator preferably has a timer, a clock or a timer.

It is possible and particularly preferred that a data processing device of the incubator is programmed to determine an occupancy state of the interior of the incubator chamber, and/or is preferably programmed to perform one or more of the following steps, in particular to determine an occupancy state of the interior of the incubator chamber as a function of the ID position data of the at least one object arranged in the interior, to determine an occupancy state of the interior of the incubator chamber as a function of the class-related

ID position data of the at least one object arranged in the interior, to determine an occupancy state of the interior of the incubator chamber as a function of the individual-related ID position data of the at least one object arranged in the interior.

An occupancy state of the interior space can be defined by information which describes the volume occupied in the interior space by the at least one object, and/or which describes the volume not occupied in the interior space by the at least one object, i.e. the free volume, and/or the storage space occupied by the at least one object on at least one storage area or on the total available storage area in the interior space of the incubator chamber, and/or the storage space not occupied by the at least one object, i.e. the free storage area, on at least one storage area or on the total available storage area in the interior of the incubator chamber, it being possible for this information to relate in each case to the total interior volume or the total storage space, it being possible for this information to include, for example, the ratio of an unavailable (occupied) or a free (unoccupied) interior volume to the total volume of the interior, or it being possible for this information to include, for example, the ratio of an unavailable (occupied) or a free (unoccupied) storage space to the total storage space in the interior.

Occupancy status data, which contains information about the occupancy status, can also contain the ID position data, class-related ID position data and/or individual-related ID position data. In this way, it is possible to specify a spatial resolution of the occupancy, i.e. the localization of the occupancy in the interior, or a density distribution of the objects in the interior.

It is possible and particularly preferred that a data processing device of the incubator is programmed to store information about the occupancy status of the incubator in the form of occupancy status data in a data memory, in particular to transfer it to an external data processing device, in particular a laboratory device, a PC or a mobile computer, in particular a tablet computer or a smartphone.

It is possible and particularly preferred that a data processing device of the incubator is programmed to display information about the occupancy status of the incubator on a screen of the incubator or an external data processing device, in particular as a function of occupancy status data which may be taken from a data memory. The external data processing device can be part of a laboratory device, PC, a mobile computer, in particular a tablet computer or a smartphone.

In test series on which embodiments of the present invention are based, it was found that the temperature profile in the incubator chamber resulting over time after the incubator door is opened due to the temperature control depends on the occupancy state of the incubator chamber. If a larger volume of the chamber interior is occupied by existing objects, there is a smaller free chamber interior volume resulting from the difference between the chamber interior and the volume occupied by the objects. In this situation, a temperature control system designed to control the entire interior volume may achieve other, undesirable results. A rapid overshoot may occur, which is undesirable, even if the recovery to the target temperature, e.g. 37° C., may be accelerated, i.e. even if the recovery time is shortened. If several new objects with lower temperatures than the target temperature are newly set, the recovery time may also be delayed, but knowledge of colder, newly set objects can also be used to adjust the temperature control. The temperature control of the temperature inside the incubator chamber depends on control parameters.

Preferably, an electronic control device of the incubator is set up or programmed so that at least one temperature control device of the incubator, which is arranged to control the temperature of the incubator chamber, is operated with the electrical power Ptemp (t) during temperature control as a function of the time t. In particular, the incubator can be set up so that the temperature control device is operated by means of pulse width modulation (PWM) of the current. The power is then determined in particular by the duty cycle of the PWM, since the amplitude of the current is preferably constant. In particular, the variables mentioned can be temperature control variables, i.e. control parameters.

Preferably, an electronic control device of the incubator is set up or programmed so that the temperature control or the control of the incubator gas supply (e.g. CO₂, N₂, and/or O₂), in particular at least one control parameter, can be adjusted depending on the occupancy status of the incubator. In this way, the influence of objects arranged in the interior of the incubator chamber on the response behavior of the controlled system can be taken into account. In particular, the recovery time can be reduced if the interior is more crowded.

The data processing device of the image capture system or other system is preferably separate from a first data processing device of the incubator. However, it can also be part of the control device of the incubator (also referred to as “first control device”), which controls functions of the incubator. The functions of the control device are implemented in particular by electronic circuits. The data processing device of the image capture system may comprise at least one CPU, and optionally at least one GPU. A GPU can be provided for image processing or the execution of deep learning processes. As an alternative to a CPU, or a GPU, the data processing device may also comprise a dedicated chip, e.g. the Nvidia Jetson, for image processing or performing deep learning processes, which may preferably be used in object tracking, in particular in a possible object classification or object individual recognition. Such dedicated chips can be added to the data processing device as computing accelerators. A GPU is already present on many System on a Chip (SoC) systems (for graphics and video playback). A Raspberry PI can also comprise a dedicated GPU unit as part of the SOC.

The object tracking system may comprise a control device, which can be provided separately from the first control device. The terms “control device” and “controlling device” are used synonymously in this description. A control device may comprise a microprocessor, which may include the data processing device. The microprocessor may be of the “Rasberry Pi” type. The control device and/or the data processing device is preferably designed to carry out a control procedure, which is also referred to as control software or control program—in each case relating to the incubator and/or the object tracking system. The functions of the incubator and/or the object tracking system and/or the control device and/or the data processing device can be described in process steps. They can be implemented as components of the control program, in particular as subroutines of the control program.

In the context of the present invention, a control device—as an optional component of the system according to the invention or of the incubator-generally comprises in particular the data processing device or the data processing device, in particular a computing unit (CPU) for processing data and/or a microprocessor, or is the data processing device. The data processing device of the control device of the incubator can preferably also be set up to control the object tracking system.

The data processing device of the image capture system is preferably a device arranged outside the incubator chamber or the incubator and in particular optionally separately from the latter, also referred to as an external device or external data processing device. The data processing device and the incubator are preferably in a data connection and are preferably components of a network for data exchange.

The at least one camera of the image capture system is preferably connected to the control device or data processing device of the image capture system via a cable connection. For this purpose, the incubator chamber has a through opening (port) through which the cable of the cable connection is guided. A seal, in particular a silicone seal, is preferably provided to seal the port in order to prevent (as far as possible) any influence on the atmosphere in the incubator. Alternatively, the camera is connected to the control device or data processing device for wireless data exchange, e.g. via Bluetooth or WLAN.

The incubator may comprise a partial housing in which, in particular, at least one control device (of the incubator and/or the object tracking system) is arranged. The partial housing is preferably arranged at the rear of the incubator, i.e. in particular opposite the incubator door.

The system, the incubator and/or the image capture system and/or the data processing device and/or the control device are preferably set up to use the position data of the at least one object or a plurality of objects to form an electronic documentation file, in which the positions and/or movement of the objects and/or their residence time and/or the identification data of the user initiating the movement in the incubator are logged and documented. This documentation file is then stored in particular in a data storage device and preferably updated continuously. In this way, “correct” handling of the objects according to standard protocols can be certified if required. On the other hand, deviations from standard protocols can be subsequently identified and/or information correlations can be determined. By recording such data, the quality of cell-based laboratory work or medical, biological and pharmaceutical processes can be significantly improved and become more reliable. The reproducibility of cell-based laboratory work can be increased, deviations from normal properties can be detected at an early stage to give the user the opportunity to correct or repeat the experiment at an early stage. The documentation file can be provided to the user or an external data processing device by the control device via data exchange. Such documentation is particularly useful in critical applications, e.g. for forensic purposes or where cells of considerable value are cultivated.

In particular, the invention also relates to a retrofit system for incubating living cell cultures comprising an incubator for incubating living cell cultures comprising:
an incubator chamber for receiving objects, in particular cell culture containers, which comprises opposite inner walls and a chamber opening for the supply and removal of the objects by a user, and which comprises at least one storage area for storing the objects, which extends between the opposite inner walls, an incubator door for closing the chamber opening, an image processing image capture system set up for retrofitting the incubator, comprising a data processing device with a data memory, a lighting device, and a camera device which is set up to capture at least one storage area of the incubator chamber, wherein the image capture system is set up, and in particular the data processing device is programmed for this purpose,
to illuminate the storage area of the incubator extending between the inner walls by means of the lighting device,
to capture at least one image of the storage area extending between the inner walls by means of the camera device, and
optionally: to store the at least one image in the data storage device in the form of image data by means of the data processing device,
to determine at least one occupancy value from the image data, which characterizes the occupancy of the at least one storage area;
to perform at least one mathematical comparison operation that compares the at least one occupancy value with at least one occupancy reference value;
to record the result of the at least one mathematical comparison operation in at least one occupancy evaluation parameter; and
to store at least one occupancy evaluation parameter in the data storage device.

The above-mentioned retrofit system is thus based on an incubator which can be retrofitted with a retrofittable image capture system, as this is an integral part of the incubator in claim 1, wherein the retrofittable image capture system must be correspondingly compatible with the “compatible incubator” so designated. “Retrofitting” preferably means in each case that the camera device can be suitably arranged or fastened in the incubator chamber, that the lighting device can be suitably arranged or fastened in the incubator chamber, that in particular the data processing device with data memory can be suitably arranged or fastened in/on the incubator, that in particular the data processing device with data memory can be suitably arranged or fastened in/on the incubator, and that in particular the data processing device with data memory can be suitably arranged or fastened in/on the incubator. that in particular the camera device and/or the lighting device and/or the data processing device with data memory are connected or can be connected to a data processing device of an incubator or an external computer for the purpose of data transmission, that in particular the camera device and/or the lighting device and/or the data processing device with data memory are connected or can be connected to a power supply which can be part of the image capture system or the incubator. It may also be provided, in particular alternatively or additionally, that the data processing device of the image capture system is formed by a data processing device of the incubator, in that the camera device and/or the lighting device are or can be connected to a data processing device of the incubator for the purpose of data transmission.

Preferably, a control device of the incubator according to the invention or of the compatible incubator, which in particular can also control the atmospheric parameters in the incubator chamber (temperature, gas partial pressure CO₂, H₂O etc.) or its data processing device is set up or programmed to determine at least one operating parameter of the incubator as a function of data from the image capture system, in particular of position data or of the final position of at least one object in the storage area, in particular a parameter which controls the display of information on a screen of the incubator or a parameter which is displayed on the screen of the incubator. the final position of at least one object in the storage area, to determine at least one operating parameter of the incubator, in particular a parameter which controls the display of information on a screen of the incubator or a parameter which is displayed on the screen of the incubator. In particular, position data or the end position of at least one object can be displayed on the screen.

Preferably, the system for incubating living cell cultures has: an external device which is in data exchange communication with the incubator, in particular a user identification device, in particular a mobile user identification device, and in particular a data exchange device by means of which the data processing device can exchange data with the external device, in particular can determine user identification data by means of the user identification device.

The invention also relates to a method for image acquisition in an incubator used for incubating living cell cultures, comprising:
an incubator chamber for holding objects, in particular cell culture containers, which comprises opposite inner walls and a chamber opening for the supply and removal of the objects by a user, and which comprises at least one storage area for storing the objects, which extends between the opposite inner walls,
an incubator door to close the chamber opening,
an image capture system, comprising
   a lighting device,
   a camera device and
   a data processing device, wherein the method comprises the steps of:
illuminating of the storage area extending between the inner walls by means of the lighting device,
capturing at least one image of the storage area extending between the inner walls during illumination by means of the camera device in the form of image data,
determining at least one occupancy value from the image data, which characterizes the occupancy of the at least one storage area;
performing at least one mathematical comparison operation that compares the at least one occupancy value with at least one occupancy reference value;
capturing the result of the at least one mathematical comparison operation in at least one occupancy evaluation parameter; and
storing the at least one occupancy evaluation parameter in the data storage apparatus.

The invention also relates to an image processing image capture system, in particular for retrofitting an incubator, comprising
a lighting device,
at least one camera device and
a data processing device with a data memory, wherein the image capture system is configured to,
to illuminate the storage area extending between the inner walls by means of the lighting device,
to capture by means of the camera device at least one image of the storage area extending between the inner walls in the form of image data, and
to determine at least one occupancy value from the image data, which characterizes the occupancy of the at least one storage area;
to perform at least one mathematical comparison operation that compares the at least one occupancy value with at least one occupancy reference value;
to record the result of the at least one mathematical comparison operation in at least one occupancy evaluation parameter; and
to store the at least one occupancy evaluation parameter in the data storage apparatus.

Further preferred embodiments of the objects according to the invention, in particular of the method according to the invention, can be taken from the description of the system with incubator according to the invention and its preferred embodiments. Further embodiment options of the invention can also be seen from the embodiment examples in the figures. Identical parts of the embodiments are essentially identified by identical reference signs, unless otherwise described or unless otherwise apparent from the context. The figures show

FIG. 1 shows a perspective view of a system according to the invention with an incubator according to an embodiment example.

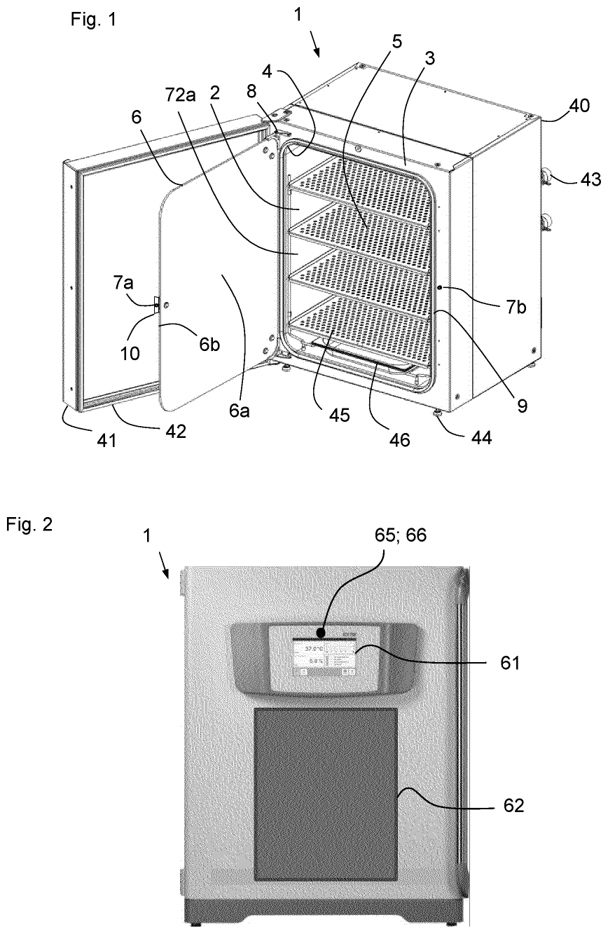

FIG. 2 shows a front view of the incubator from FIG. 1.

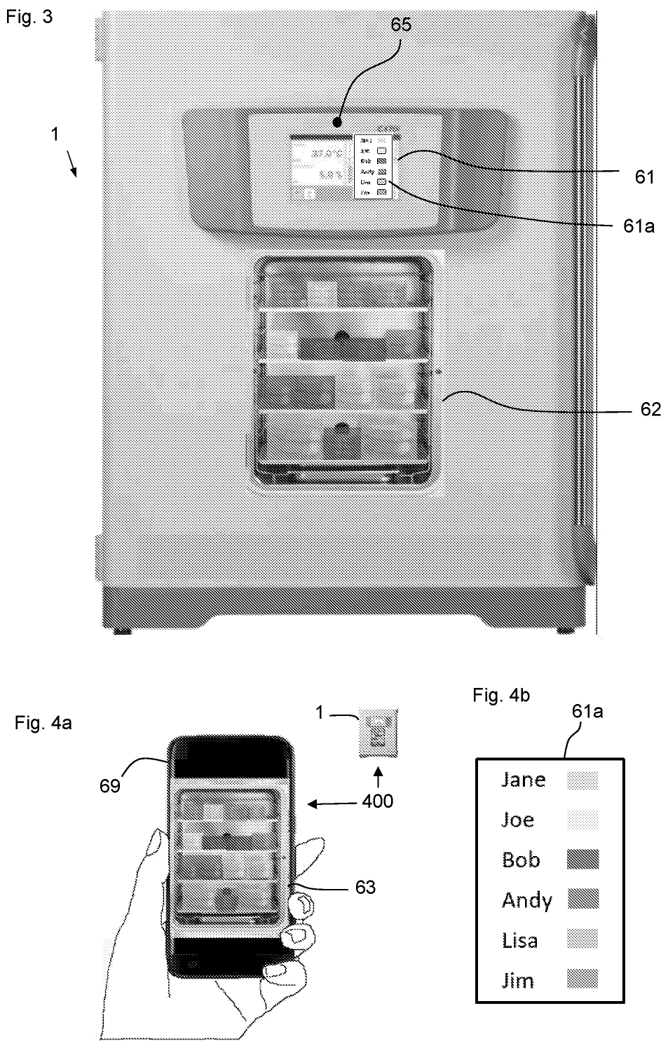

FIG. 3 shows a front view of the incubator from FIG. 1 with a graphic representation of the occupancy of the incubator chamber with objects that are highlighted in color-coded form according to the user.

FIG. 4a shows a smartphone with camera and display 63 as an external device, which can be part of a system 400 comprising the incubator 1 of FIG. 3 and the smartphone 69.

FIG. 4b shows a legend of the color coding used in the screen of FIG. 3 to highlight user-related objects.

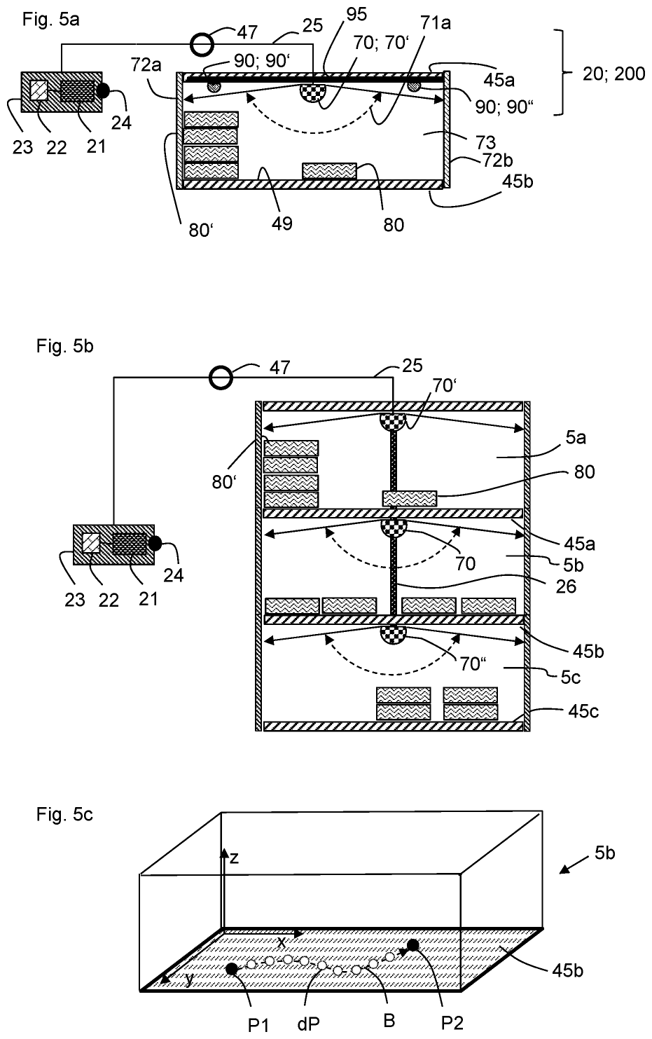

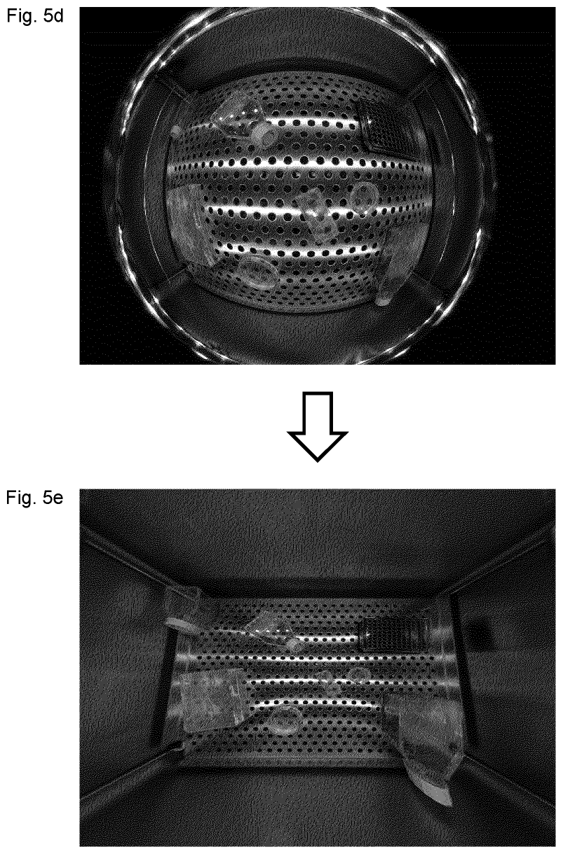

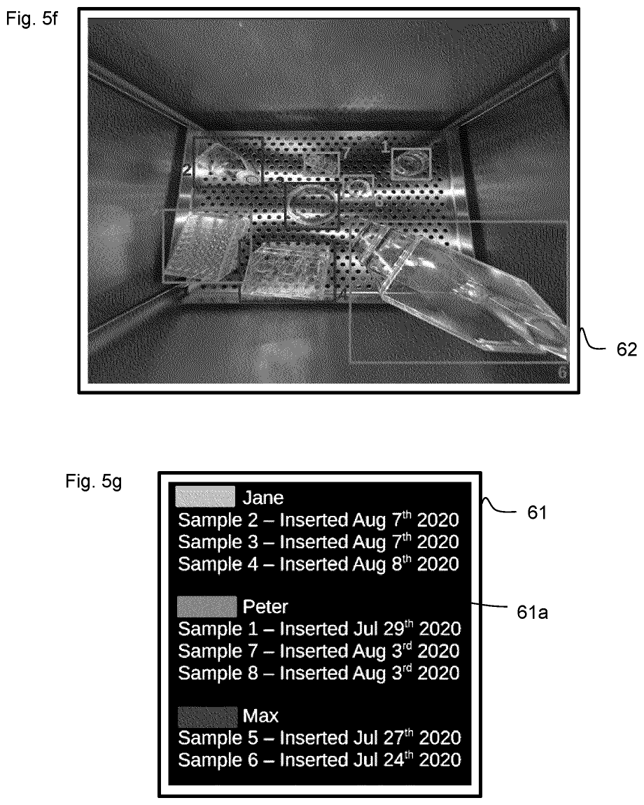

FIG. 5a shows a schematic side view of an image capture system as a component of the incubator of FIGS. 1 to 4b, in an example of a chamber with a single monitored bearing plate.

FIG. 5b shows a schematic side view of an image capture system as a component of the incubator of FIGS. 1 to 4b, in an example of a chamber with several monitored bearing plates.

FIG. 5c shows a perspective view of a storage area monitored by the object tracking system of FIGS. 5a and 5b, as well as the start position P1, position changes dP and end position P2 of a tracked object in relation to a coordinate system.

FIG. 5d shows a digital image captured by the wide-angle fisheye camera of the image capture system used in FIGS. 5a and 5b, which appears distorted due to the optics.

FIG. 5e shows the image of FIG. 5d, which has been rectified by the image capture system using straightening algorithms.

FIG. 5f shows a still image captured by the wide-angle fisheye camera of the image capture system used in FIGS. 5a and 5b for output to a screen of the incubator, showing the bounding boxes of the image capture system, identification numbers and color coding identifying the user/owner.

FIG. 5g shows a possible screen content that can be displayed on a screen of the incubator to explain the screen page shown in FIG. 5f.

FIG. 6 shows a schematic top view of a storage area of the incubator from FIGS. 1 to 5f, including objects, which is arranged in an image capture section of a camera of the image capture system.

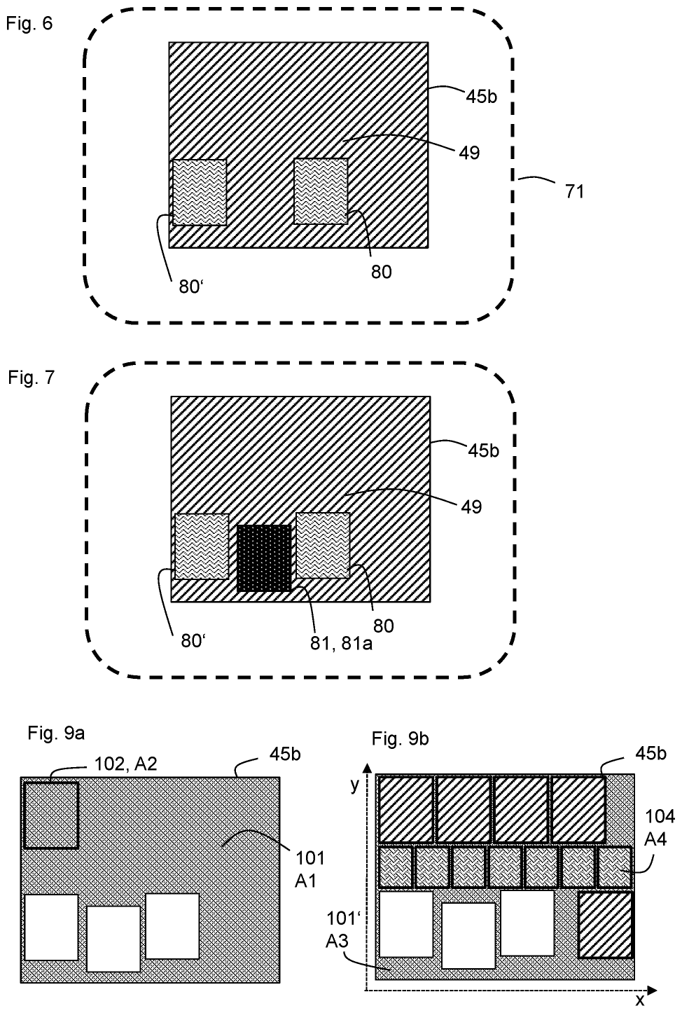

FIG. 7 uses the section from FIG. 6 to show the detection of a new object placed between two existing objects in the incubator.

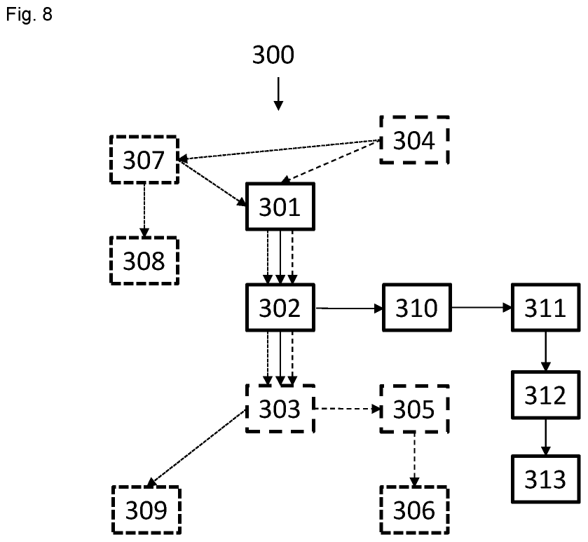

FIG. 8 schematically shows the sequence of an exemplary process according to the invention.

FIGS. 9a, 9b show the image evaluation for carrying out a comparison operation and determining an occupancy value parameter by means of segmentation, as an example of the invention, using the occupancy of the storage area in FIG. 7.

FIG. 1 shows an incubator 1 for storing laboratory samples, more specifically a CO₂ incubator for storing living cell cultures in a defined atmosphere at a controlled temperature, e.g. 37° C. For this purpose, the chamber interior 5 of the incubator is thermally insulated and can be sealed off from the environment in a gas-tight manner; the gas composition in the interior is also regulated and can be changed via gas connections 43. The chamber housing 2 of the incubator stands on bases 44, encapsulates the interior 5 and opens into the front 3 of the incubator. The front side has the chamber opening 4, through which the chamber interior 5 is accessible. A transparent inner chamber door 6 is used to close the chamber opening when the chamber door is in a closed position. In the incubator 1, the chamber housing 2 is placed within the interior of an outer housing 40, so that the chamber housing 2 and the outer housing 40 are spaced apart and thermally insulated from each other. Shelf inserts 45 and a humidifier tray 46 can be seen in the chamber interior. The front side 3 of the chamber housing and the front side of the outer housing coincide in the present case.

The outer incubator door 41 and the chamber door 6 are shown in an open position. The outer door 41 is hinged to the outer edge of the outer housing and has a circumferential seal, in particular silicone seal 42.

When the outer door 41 has been opened, the inner chamber door 6 of the incubator is initially still closed. The locking device (10, 7a, 7b) is used for this purpose. With the chamber door 6 closed, the user can first view the interior 5 through the transparent door wall before opening the door and inserting or removing a laboratory sample. Nevertheless, opening the outer incubator door 41 already represents a disturbance that can potentially damage the incubator atmosphere.

The incubator comprises a forward-facing external camera 65 built into the door 41, the images of which can be evaluated by the suitably programmed data processing device of the incubator, in particular in order to identify a user by means of facial recognition, wherein the external camera 65 connected to the data processing device serves as a user identification device 66. The latter can also be carried out via the camera of the smartphone 69.

In order to protect the stored laboratory samples, it is effective for the incubator to minimize the time during which the interior of the incubator is exposed to the environment (opening time intervals). The present invention is based on the observation that the opening time intervals can be reduced by an image capture system 200. The incubator 1 has an image capture system (not shown in FIGS. 1, 2).

As shown in FIG. 2, the outside of the outer incubator door has a first screen, a touchscreen 61, via which operating parameters of the incubator 1 are displayed, e.g. the temperature of the incubator atmosphere or a gas partial pressure in the interior 5.

The outside of the outer incubator door 41 has a second screen 62, which may be a touch screen. However, instead of a second screen, all screen outputs may be provided on a single screen. The data processing device (not shown) of the incubator 1 is programmed to display on the screen 62 the occupancy of the interior of the incubator. The screen 62 serves as a “digital window” that allows the user to take a (virtual) look inside the incubator. The graphical representation of the interior or the at least one storage area of the incubator and its occupancy with stock objects can be programmed in such a way that certain stock objects are graphically highlighted depending on certain criteria or condition parameters.

Here, the system according to the invention essentially comprises the incubator 1, in which the data storage apparatus and the data processing apparatus, which perform the functions, are installed:
to determine at least one occupancy value from the image data, which characterizes the occupancy of the at least one storage area;
to perform at least one mathematical comparison operation that compares the at least one occupancy value with at least one occupancy reference value;
to record the result of the at least one mathematical comparison operation in at least one occupancy evaluation parameter; and
to store the at least one occupancy evaluation parameter in the data storage device.

Regarding FIG. 3: The data processing device of the incubator 1 is also programmed here to display one or more objects in the display 62 depending on at least one condition parameter, which here depends on user identification data, according to their respective position in the interior of the incubator, which was determined by means of the image capture system. In each case, the inventory objects associated with specific user identification data identifying a specific user are highlighted with a specific user-dependent color. The legend 61a for this type of color coding is shown to the user here via the upper display 61 in its sub-area 61a. The legend 61a is shown larger in FIG. 4b: the user IDs “Jane”, Joe” etc. are assigned the corresponding highlighting colors used in the display 62, 63.

FIG. 4 shows that the output display 61 and/or 62 can also—alternatively or additionally—be component(s) of an external device, here a smartphone 69, which is in a data exchange connection with the incubator and comprises the display 63, which here functions as a component of an incubator system.

FIG. 5a shows a schematic front view of the shelf inserts 45a and 45b of the incubator 1 as bearing plates for objects, which are arranged one above the other. The vertical distance between such shelf inserts 45 in incubators is usually not large and is, for example, between 10 and 40 cm, in the case of incubator 1 approximately 15 cm. As a result, either several cameras must be used in order to capture the entire storage area 45, in this case the entire storage space of the shelf insert 45b and the “air space” above it up to the shelf insert 45a. The camera 70 or camera device 70′ is or includes a wide-angle or wide-angle fisheye optical camera with a diagonally measured image angle of approximately 200°.

FIG. 5a shows the image capture system 20 installed in the incubator 1, which is also designated by the reference sign 200 in the case of the design as a retrofit system. The image capture system 20 includes the camera 70, a wide-angle fisheye camera which, in the viewing range or viewing angle 71a of preferably 160° to 220°, captures the storage area of the shelf plate insert 45b located below it and a large part of approximately 80% of the areas of the incubator inner wall sections 72a, 72b which, with the bearing plates 45a and 45b extending between the inner walls 72a and 72b, delimit the compartment 73 of the incubator chamber. The wide angle of view makes it possible to use a single camera to capture the entire storage area of the shelf plate insert 45b below, in particular also the air space into which the (inventory) objects 80′ and 80 protrude, namely a stack 80′ of cell culture containers and a cell culture container 80. The nominal angle of view of the wide-angle fisheye camera is 200° here, but only an image area is evaluated which corresponds to an angle of view taken from the range of preferably 160° to 170°.

The camera is arranged vertically above the geometric center of the storage space of the shelf insert 45b. The image capture system 20 also includes the lighting device 90, the control device 23, which comprises a data processing device 21 and a data memory 22 as further components of the image capture system 20. The data processing device 21 or the control device 23 are connected to the camera 70 and the other cameras not shown in FIG. 5a via a cable connection 25, which enters the incubator chamber through the port 47 in the rear wall of the incubator chamber, each of which is provided so that all storage areas (all upper sides of shelf inserts 45, see FIG. 1) are monitored. The control device 23 also has a data interface 24, via which a data connection to further incubator device components is made possible, e.g. in order to output data or signals to a display 61, 62, 63 of the incubator. A lighting device 90 with several LEDs 90′, 90″ is mounted above the bearing plate 45b and connected to the control device 23 via lines 25. Instead of the two LEDs shown, a plurality of LEDs can be provided. By means of the optional lighting device 90, the bearing area 45b can be illuminated for the purpose of image acquisition, if this is appropriate.

FIG. 5b shows a schematic side view of an image capture system as a component of the incubator of FIGS. 1 to 4b in an example of a chamber with several monitored bearing plates 45a, 45b, 45c. The illustration is an extension of the principle of FIG. 5a, in which the incubator chamber is divided into several compartments 5a, 5b and 5c, which are arranged here one above the other and are connected for gas exchange, which is formed via holes in the bearing plates 45a, 45b, 45c. The bearing area or bearing plate 45a in compartment 5a is monitored by camera 70′, the bearing area or bearing plate 45b in compartment 5b is monitored by camera 70, and the bearing area or bearing plate 45c in compartment 5c is monitored by camera 70″, wherein cameras 70′ and 70″ are designed and arranged in the same way as camera 70 in FIG. 5a. All cameras are connected to the control device 23 via a connecting cable bundle 26 inside the incubator chamber, which merges into the cable connection 25 already shown in FIG. 5a and leaves the incubator chamber through the port 47 in the rear wall of the incubator chamber, the data processing device 21 of which is set up to monitor all objects in all three compartments 5a, 5b and 5c. The image capture system 20 of the incubator belonging to FIG. 5b here comprises three cameras 70, 70′, 70″, the lighting device 90, the data processing device 21, the data storage device 22 and the connecting lines.

FIG. 5c shows a perspective view of a compartment 5b or storage area 45b, which is monitored by the image capture system of FIGS. 5a and 5b, as well as the object positions P1, P2 referred to a Cartesian coordinate system (x, y, z). In the event that the image capture system is designed as an object tracking system by capturing video data and using digital image processing means, changes in the position dP of an object moving along its movement path B can also be tracked. The origin of the coordinate system can be fixed in a corner of the compartment.

FIG. 5d shows a digital image captured by the wide-angle fisheye camera of the image capture system used in FIGS. 5a and 5b, which appears distorted due to the optics.

FIG. 5e shows the image of FIG. 5d, which has been rectified by the image capture system using straightening algorithms.

FIG. 5f shows a still image captured by the wide-angle fisheye camera of the image capture system used in FIGS. 5a and 5b for output to a screen of the incubator, showing the bounding boxes of the image capture system, identification numbers and color coding identifying the user/owner.

FIG. 5g shows a possible screen content that can be displayed on a screen of the incubator to explain the screen page shown in FIG. 5f. In addition to the identification of the objects by identification numbers, a color coding identifying the user/owner is also shown, as well as the optional times registered by the incubator for placing the objects in the incubator chamber.

FIG. 6 shows a storage area, namely the top of the sheet metal shelf insert 45b, from a bird's eye view or top view. The image capture section 71 captured by the camera 70 is also shown schematically. The image capture section 71 is the area that is captured by the camera 70 in one or in each image, because the camera 70 does not change its angle of view or its position here. Thus, each image shows this section 71. In the figures, the lower edge of the section 71 represents the area located near the incubator chamber opening 4. However, the camera 70 and or the lighting device 90 with the two light sources (LEDs) 90′ and 90″ can also be movably or displaceably mounted by means of the transport device 95, in this case a motorized rail system.

The image capture system 20, 200 is configured to,
to illuminate the storage area 49 extending between the inner walls 72a, 72b by means of the lighting device 90,
to capture at least one image of the storage area 49 extending between the inner walls 72a, 72b by means of the camera device 70, and
to store the at least one image by means of the data processing device 21 in the form of image data in the data storage device 22.

The image capture system is also configured to,
to illuminate at least two objects 80, 80′ arranged on this storage area 49 by means of the lighting device 90,
to capture an image of the at least two objects 80, 80′ on this storage area 49 by means of the camera device 70, i.e. an image on which these at least two objects are shown, and
to store the image of the at least two objects 80, 80′ and the storage area 49 in the form of image data in the data storage device 22 by means of the data processing device 21.

The data processing device is optionally programmed for this purpose,
to distinguish the first object 80 and second object 80′ shown in the image by means of evaluation of the image data (in particular: to assign different identification data to the first and second object; to count the objects; to recognize the object class; to analyze, store, recognize individual features; to track objects in motion), in particular to detect the outlines of the first 80 and second object 80′ in the image by means of image processing algorithms, and
in particular to store information about the first 80 and second object 80′, in particular the outlines of the first 80 and second object 80′, in the form of object data in the data storage device 22.

The lighting device 90 is here optionally operable and the data processing device is here optionally programmed to operate the lighting device 90 in at least two different illumination modes, and the image capture system 20, 200 is arranged to do so,
the storage area 49 of the incubator chamber by means of the lighting device 90
   initially in a first illumination mode, in particular with active LED 90′ and inactive LED 90″, and
   then illuminated in a different second illumination mode, in particular with active LED 90″ and inactive LED 90′,
to capture at least one image of the storage area 49 during illumination by means of both the first and second illumination modes by means of the camera device 70, and
to provide the at least one image in the form of image data containing combined image information captured during both the first and second illumination modes, wherein the data processing device is programmed to execute an image evaluation program that extracts the combined image information from the image data.

Preferably, the at least one image of the storage area 49 includes at least a first image of the storage area 49 and a second image of the storage area 49 different therefrom, wherein the first image is captured in the first illumination mode and the second image is captured in the second illumination mode, and the first image is provided in the form of first image data and the second image is provided in the form of second image data, wherein the data processing device and the image evaluation program are programmed such that
the first image data and the second image data are combined to obtain combined image data, which is obtained in particular by an addition and/or averaging of first and second image data, and
the combined information is obtained from the combined image data.

The data processing device of the image capture system 20, 200 is programmed to capture and evaluate the image data by means of the camera 70 during illumination, depending on the detection of the closed state of the outer door 41 of the incubator. By comparing successive images, it is possible to determine whether a new object 81 has entered the camera section 71.

FIG. 7: An image containing an outline 81a which has newly appeared in the cutout 71 and which can be assigned to the object 81 introduced into the interior is regarded as a modified image. Based on this modified image, identification data is assigned to the newly appeared outline 81a on the assumption that it is a new object 81 to be placed in the incubator.

FIG. 8 shows the sequence of the method according to the invention, which was already indirectly described in the above description of the previous figures.

The method 300 is for capturing images in an incubator, which is for incubating living cell cultures, and comprises:
an incubator chamber for receiving objects, in particular cell culture containers, comprising opposing inner walls and a chamber opening for the supply and removal of the objects by a user, and comprising at least one storage area for storing the objects, which extends between the opposing inner walls,
an incubator door to close the chamber opening,
an image capture system, comprising
a lighting device,
a camera device and a data processing device with a data memory, wherein the method 300 comprises the program-controlled steps:
illuminating the storage area extending between the inner walls by means of the lighting device, (301)
capturing at least one image of the storage area extending between the inner walls during illumination by means of the camera device, (302)
optional: storing the at least one image in the form of image data in the data storage device, (303)
determining at least one occupancy value from the image data, which characterizes an occupancy of the at least one storage area; (310)
performing at least one mathematical comparison operation comparing the at least one occupancy value with at least one occupancy reference value; (311)
capturing the result of the at least one mathematical comparison operation in at least one occupancy evaluation parameter; (312) and storing the at least one occupancy evaluation parameter in the data storage apparatus (313).

In particular, the step 310 for determining the occupancy value A1 preferably includes at least one of the following steps, which may be performed repeatedly if required:
determining of a contiguous free area segment 101 (area not occupied by an object), optionally determination of several contiguous free area segments by means of computer-aided image processing, in particular using segmentation;
determining the area A1 (e.g. measured in cm²) of segment 101;
determining a contiguous free area segment 101 which is larger than an area A2, optionally: determining several contiguous free area segments, each of which is larger than an area A2, by means of computer-aided image processing, in particular using segmentation;
determining a contiguous free surface segment 101 which is larger than a surface A2 and whose shape corresponds to a predefined dimension, in particular a suitable footprint A2 with a suitable dimension for placing a laboratory sample container. The suitable dimension M may be taken from a group of different suitable dimensions M, which may be stored in the data storage apparatus. A suitable dimension is assigned to one of several laboratory sample containers, e.g. an SBS standard microtiter plate, cell culture bottles of different sizes, Petri dishes of different sizes.

In particular, the step 311 of performing a mathematical comparison operation comparing the at least one occupancy value with at least one occupancy reference value includes the following step:
Use the mathematical comparison function V=V (A1; A2) to determine whether the following applies to the areas A1 and A2: A1>=A2.

The step 312 of capturing the result of the at least one mathematical comparison operation in at least one occupancy evaluation parameter includes, in particular, the following step:
Determine the occupancy evaluation parameter PBB=V=(0 or 1), which here is of the Boolean type, where PBB=1 if the area A1 is greater than or equal to the area A2, and where PBB=0 if the area A1 is less than the area A2.

In FIG. 9a, the situation from FIG. 7 is shown on an image 71 of the storage area 45b, which is evaluated using computer-aided image processing methods. Segmentation is used to recognize the area segment 101 that is not occupied by a cell culture container 80′, 81, 80. The area of this surface segment 101 is determined as A1. The total area of the storage area can be divided into a grid for computational processing; positions of the grid points can be recorded in a Cartesian coordinate system (x; y). A grid area or grid point can be assigned the occupancy attribute BA (occupied or free). An occupied area with a specific dimension can be assigned to an object. A free area with a specific dimension A2 can be assigned to a free footprint 102. Free footprints 102, 104 can be successively compared by determining the free segment area 101, 101′ and comparing it with at least one value A2 or A4 and a suitable dimension (FIG. 9b). This automatic allocation can be continued until the area of the storage area 45b is optimally utilized by footprints 102, 104, . . . , which can be noted as “free, reserved” or “occupied”. Such automated planning can be used to implement a user guidance system.

As an alternative to a dynamic allocation of footprints 102, 104 to the area of the storage area, a grid with predefined footprints could also be used, which are then recognized as “free” or “occupied” by image capture.

Preferably, the steps are also carried out in the method 300:
monitoring, over time, of the incubator chamber 2, 5 by means of at least one camera 70 of the camera device 70′ of the incubator, which is arranged to record at least one storage area 49 in the interior of the incubator chamber, into which the at least one object 80; 80′; 81 is placed; (304)
assigning identification data to the at least one object 80; 81 captured in an image of the storage area 49 taken by the at least one camera 70 after positioning thereof in the storage area; (305)
storing the position of the at least one object 80; 81 in the storage area 49 in dependence on the identification data of the at least one object as ID position data, in the data memory. (306)

Preferably, the method 300 further includes the steps of:
reading user identification data identifying the user of the incubator 1 who introduced the at least one object 80; 81 into the incubator chamber by means of a user identification device (307), and
storing the user identification data in a data memory of the incubator. (308)

Preferably, the method 300 also includes the step of:
saving the ID position data depending on the user identification data as user-related ID position data. (309)

1. A system for monitoring the occupancy of storage place in at least one incubator for incubating living cell cultures, comprising:
at least one incubator for incubating living cell cultures, comprising:
an incubator chamber for holding objects, in particular cell culture containers, which comprises opposite inner walls and a chamber opening for the supply and removal of the objects by a user, and which comprises at least one storage area for storing the objects, which extends between the opposite inner walls,
an incubator door to close the chamber opening,
an image capture system comprising:
a lighting device,
at least one camera device and
a data processing device,
wherein the image capture system is configured to
illuminate the at least one storage area extending between the inner walls by means of the lighting device,
using the camera device to capture at least one image of the at least one storage area extending between the inner walls in the form of image data,
wherein the system comprises a data storage apparatus and a programmable data processing apparatus, programmed thereto:
to determine at least one occupancy value from the image data, which characterizes the occupancy of the at least one storage area;
to perform at least one mathematical comparison operation that compares the at least one occupancy value with at least one occupancy reference value;
to record the result of the at least one mathematical comparison operation in at least one occupancy evaluation parameter; and
to store the at least one occupancy evaluation parameter in the data storage apparatus.

2. The system according to claim 1, wherein the data processing apparatus is programmed thereto:
to determine from the image data, as an occupancy value, a preferably contiguous sub-area of a storage area of the at least one storage area, which in particular is not occupied by objects, and to determine the size of this free storage space.

3. The system according to claim 2, wherein the data processing apparatus is programmed thereto:
to perform the mathematical comparison operation in which the size of the free storage space is compared with a reference value of a free storage space.

4. The system according to claim 3, wherein the free storage space is the footprint of a storage location for a laboratory sample container, and wherein the occupancy reference value is a predetermined reference value suitable for this footprint.

5. The system according to claim 4, wherein the data processing apparatus is programmed thereto:
to assign storage location ID data in particular to the storage location, which uniquely identifies this storage location from other storage locations.

6. The system according to claim 4, wherein the data processing apparatus is programmed thereto:
to determine the availability of a storage location depending on the occupancy evaluation parameter, i.e. to answer the question of whether the storage location is free or occupied, and in particular to store the answer to this question as occupancy evaluation data.

7. The system according to claim 1, wherein the data processing apparatus is programmed thereto:
to define occupancy evaluation data depending on the occupancy evaluation parameter
to output information about the occupancy evaluation data to the user via a user interface device.

8. The system according to claim 7, wherein the occupancy evaluation data information is one of the following:
the number of free storage locations for laboratory sample vessels of a predetermined storage area determined using the mathematical comparison operation;
the number of occupied storage locations for laboratory sample vessels of a predetermined storage area determined using the mathematical comparison operation;
exceeding or falling below a threshold value that characterizes underutilization or overutilization of the at least one storage area of the incubator.

9. The system according to claim 1, wherein the data processing apparatus is programmed thereto:
to generate incubation report data for an individual sample contained in an individual laboratory sample container by assigning the laboratory sample container a sample ID that uniquely identifies the laboratory sample container and by assigning the laboratory sample container a storage location ID.

10. The system according to claim 1, comprising a camera apparatus, which can in particular be the camera device of the image capture system, by means of which at least one image of at least one storage location of at least one storage area can be captured from the image data, in particular in order to capture an image documentation of the storage location.

11. The system according to claim 1, comprising a positioning guidance system comprising a lighting device by means of which an area or spot in the storage area, in particular a storage location, can be illuminated in a targeted manner.

12. The system according to claim 11, wherein a data processing device of the incubator, or the data processing apparatus, is programmed to set the target of illumination in dependence on sample ID data and or storage location ID data and to apply directional illumination to that target or storage location.

13. The system according to claim 1, comprising a user guidance system comprising a lighting device and configured to support the positioning of laboratory sample containers on- and/or between-storage locations of the incubator by illuminating at least one storage location and/or a laboratory sample container placed on a storage location according to a predetermined schedule.

14. The system according to claim 1, comprising two or more incubators connected to each other for the purpose of data exchange, each of the incubators comprising:
an incubator chamber for holding objects, in particular cell culture containers, which comprises opposite inner walls and a chamber opening for the supply and removal of the objects by a user, and which comprises at least one storage area for storing the objects, which extends between the opposite inner walls,
an incubator door to close the chamber opening,
an image capture system comprising:
a lighting device,
at least one camera device and
a data processing device,
wherein the image capture system is configured to,
illuminate the at least one storage area extending between the inner walls by means of the lighting device,
capture at least one image of the at least one storage area extending between the inner walls in the form of image data by means of the camera device,
wherein the system comprises a data storage apparatus and a programmable data processing apparatus programmed thereto:
to determine from the image data of each of the at least two incubators in each case at least one occupancy value which characterizes an occupancy of the at least one storage area of the respective incubator;
to perform at least one mathematical comparison operation that compares the at least one occupancy value with at least one occupancy reference value;
to record the result of the at least one mathematical comparison operation in at least one occupancy evaluation parameter; and
to store the at least one occupancy evaluation parameter in the data storage apparatus.

15. A method of image acquisition in an incubator used for incubating living cell cultures, comprising:
an incubator chamber for holding objects, in particular cell culture containers, which comprises opposite inner walls and a chamber opening for the supply and removal of the objects by a user, and which comprises at least one storage area for storing the objects, which extends between the opposite inner walls, an incubator door to close the chamber opening,
an image capture system, comprising:
a lighting device,
a camera device and
a data processing device with a data memory,
wherein the method comprises the steps of:
illuminating of the storage area extending between the inner walls by means of the lighting device,
capturing at least one image of the storage area extending between the inner walls during illumination by means of the camera device in the form of image data,
determining at least one occupancy value from the image data, which characterizes the occupancy of the at least one storage area;
performing at least one mathematical comparison operation that compares the at least one occupancy value with at least one occupancy reference value;
capturing the result of the at least one mathematical comparison operation in at least one occupancy evaluation parameter; and
storing the at least one occupancy evaluation parameter in the data storage apparatus.
